(19) United States
(12) Patent Application Publication
(10) Pub. No.: US 2018/0120278 A1
(43) Pub. Date: May 3, 2018

(54) APPARATUS FOR VOLATILE ORGANIC COMPOUND (VOC) DETECTION

(71) Applicant: THE UNIVERSITY OF BRITISH COLUMBIA, Vancouver (CA)

(72) Inventors: Mina HOORFAR, Kelowna (CA); Mohammad PAKNAHAD, Kelowna (CA); Ali AHMADI, Kelowna (CA)

(21) Appl. No.: 15/800,679

(22) Filed: Nov. 1, 2017

Related U.S. Application Data
(60) Provisional application No. 62/415,640, filed on Nov. 1, 2016.

(51) Int. Cl. G01N 33/00; G01N 27/12

(52) U.S. Cl. CPC G01N 33/0047; G01N 27/12

(57) ABSTRACT

Provided is an apparatus for the detection of volatile organic compounds (VOCs) for biological analysis, environmental testing and analytical testing. The gas detection apparatus includes: a channel having an inner surface and having at least one opening, such that the channel is optionally in fluid communication with a sample gas, the inner surface having a coating comprising: a first layer comprising a non-reactive metal or non-reactive metalloid compound; a second layer comprising a moisture barrier with high porosity; and a gas sensor disposed within the channel. Embodiments described herein provide low cost and highly selective gas detectors.

### CROSS REFERENCE TO RELATED APPLICATION

This application claims priority from U.S. Provisional Application Ser. No. 62/415,640 filed 1 Nov. 2016.

### TECHNICAL FIELD

The present invention provides an apparatus for detecting and differentiating volatile organic compounds (VOC) produced from a gas or liquid sample. In particular, this invention relates to gas detection apparatus having a coated channel and a gas sensor.

### BACKGROUND

There is a need for rapid, sensitive and high precision detectors of volatile organic compound (VOC) gases for different applications including beverage and food quality assessment [1], analytical chemistry [2], biological diagnosis [3-5], and safety and environmental monitoring [6]. Numerous approaches have been developed, for detection of VOCs. Gas chromatography (GC) [7] and mass spectrometry (MS) [8] are the most commonly used methods, which provide high sensitivity and selectivity. However, miniaturization of these methods, which is required for numerous emerging applications [9-10] is challenging due to the complexity of their fabrication, calibration and sample extraction processes. Moreover, their high cost and long processing time hinder the implementation of these techniques to applications, which require disposable and rapid detection methods [11].

More recently, electronic nose (e-nose) systems, have been used as an alternative method of gas detection. E-nose systems, are based on sensor arrays coupled with pattern recognition systems. In an e-nose system, the gas sensor array provides a fingerprint response to a given odor; then, a pattern recognition software tool, is used to perform odor identification and discrimination [12-13]. Despite the general success of electronic noses, there are practical challenges in adaptation of this technology: in essence, the inevitable multidimensional drifts of the components of the sensor array result in frequent replacement of the expensive parts and cumbersome recalibrations [14]. Moreover, since general-purpose gas sensors are not selective against different gases, the sensor array used in e-noses is required to have a specific sensor for detecting each target gas. This makes the drift compensation and sensor recalibration even more complicated [15-16].

Recently, microfluidic-based gas detectors with high selectivity and sensitivity features of both traditional methods (GC and MS) and e-noses have been introduced [17-21]. These systems function based on analyzing the kinetic response of diffused gases in micro-channels using a single general purpose gas sensor [18-21]. As each gas has different diffusion and physical adsorption rates, microfluidic-based gas detectors successfully differentiate among the components of a mixture (and even binary mixtures of different isomers) [20]. Although these devices are selective to different gases, they cannot differentiate among components of complex mixtures at low concentrations. Moreover, due to the slow process of gas diffusion in the microchannels and also chemical adsorption of gas molecules to the channel walls, the recovery process of fabricated sensors takes relatively long time (up to 10 minutes) [20]. It has been recognized that the diffusion constants of a target gas depends on the temperature of the diffusion medium [29] and clearance of a channel may be accomplished by providing flow of air or a pure gas in the opposite direction of the diffusion process [29]. However, the design of microfluidic-based gas detectors must be further optimized to improve their performance.

### SUMMARY

The present invention is based in part on the discovery that different channel coating materials can have a beneficial effect the performance of the microfluidic-based gas detectors. In particular, numerous different coating combinations for the channel were compared. Moreover, the geometry of the channel was optimized to study the effect of channel dimensions on the selectivity and recovery time of the device. To show the diagnostic power of the developed miniaturized gas detector, in terms of differentiating small concentrations (ppm level) of different volatile organic compounds (VOCs), a range of different target gases including alcohol and ketone vapors; methanol and tetrahydrocannabanol (THC) were tested and successfully differentiated. As described herein, the selectivity of microfluidic gas detectors may be significantly enhanced by optimizing the micro-channel geometry and surface treatment. Moreover, the sensor recovery time may be reduced to 150 seconds, which is significantly faster than the recovery time reported in previous studies [20]. Furthermore, the integration of heaters along the micro-channels to enhance the diffusion rate of the THC molecules in the channel and decreasing the sensor response and recovery time to below 200 s. Accordingly, the improvements described herein may advance the state-of-the-art gas analysis methods, but especially for applications [22] requiring real-time sensing.

In accordance with a first embodiment, there is provided a gas detection apparatus, the apparatus including: (a) a channel having an inner surface and having at least one opening, such that the channel may be in fluid communication with a sample gas through the opening, the inner surface having a coating including: (i) a first layer comprising a non-reactive metal or non-reactive metalloid compound; (ii) a second layer comprising a moisture barrier; and (b) a gas sensor disposed within the channel.

In accordance with a further embodiment, there is provided a gas detection apparatus, the apparatus including: (a) a channel having an inner surface and having at least one opening, such that the channel may be optionally in fluid communication with a sample gas when the opening is in an open position and optionally not in fluid communication when the opening is in a closed position, the inner surface may have a coating including: (i) a first layer comprising a non-reactive metal or non-reactive metalloid compound; (ii) a second layer comprising a moisture barrier; and (b) a gas sensor disposed within the channel.

In accordance with a further embodiment, there is provided a gas detection apparatus, the apparatus including: (a) a channel having an inner surface and having at least one opening, such that the channel may be optionally in fluid communication with a sample gas when the opening is in an open position and an optional closed position, the inner surface may have a coating including: (i) a first layer comprising a non-reactive metal or non-reactive metalloid compound; (ii) a second layer comprising a moisture barrier; and (b) a gas sensor disposed within the channel.

In accordance with a further embodiment, there is provided an apparatus comprising the gas detection apparatus described herein for use in a Tetrahydrocannabinol (THC) breathalyzer.

In accordance with a further embodiment, there is provided an apparatus comprising the gas detection apparatus described herein for use in natural gas leakage detection.

In accordance with a further embodiment, there is provided an apparatus comprising the gas detection apparatus described herein for use in nuisance sewer gas detection.

The second layer may include a moisture barrier has a gas permeability sufficient to absorb the gas particles being sampled. The non-reactive metal may be selected from one or more of the following: copper; chromium; ruthenium; rhodium; palladium; gold; silver; osmium; iridium; platinum; titanium; niobium; tantalum; bismuth; tungsten; tin; nickel; cobalt; manganese; and zinc; or (ii) may be metalloid compound is SiO₂. The moisture barrier with high porosity may be Parylene or Polydimethylsiloxane (PDMS). The Parylene may be selected from Parylene C, Parylene N or Parylene D. The Parylene may be Parylene C. The non-reactive metal may be selected from one or more of the following: copper; chromium; ruthenium; rhodium; palladium; gold; silver; iridium; platinum; titanium; niobium; and tantalum. The coating may be chromium, gold and Parylene C. The channel may further include a heater. The heater may be operable to increase the channel temperature to at least 80° C. The heater may be one or more wires, one or more sputtered electrodes, one or more heating pads, or heat may be applied via optical heating, microwave heating, electromagnetic heating, combinations thereof etc. The gas sensor may be a Metal Oxide Semiconductor (MOS). The gas sensor may be a tin oxide-based chemoresistive gas sensor. The gas sensor may be an infra-red (IR) sensor. The gas sensor may be an optical sensor. The gas sensor may be a photoionization detector (PID). The gas sensor may be a chemoresistive sensor. The gas sensor may be a Metal Oxide Semiconductor (MOS), an infra-red (IR) sensor, a chemoresistive sensor, an electrochemical sensor, an optical sensor, a capacitive sensor, a semiconductor sensor, an acoustical sensor, a thermoelectric sensor, a combination of sensors, etc. There may be more than one gas sensor in the channel. There may be a pluralitiy of channels with one sensor per channel. There may be a pluralitiy of channels with more than one gas sensor in the channel. The channel length to channel depth ration may be 150:1. The channel width to channel depth ration may be 3:1. The channel length may be 3 mm wide, 30 mm long and 200 μm deep. The first layer may include chromium and gold. The chromium may be applied to the channel prior to the gold. The second layer may include Parylene C. The first layer may include SiO₂. The second layer may include Parylene C. The opening may further include a closed position. The opening may further include a open position. The opening may include an open and a closed position. The apparatus may further include a second opening. The second opening may have both an open and closed position.

The apparatus may further include a liquid trap positioned in fluid communication with the at least one opening. The apparatus may further include a humidity filter positioned in fluid communication with the at least one opening. The apparatus may further include may further include a pump which may optionally be in fluid communication with the second opening. The apparatus may further include a compressed air source, which may optionally be in fluid communication with the channel. The apparatus may further include a compressed gas source, which is optionally in fluid communication with the channel. The apparatus may further include a pentane plume, which may optionally be in fluid communication with the channel. The apparatus may further include a compressed O₂ source or N₂ source or separate O₂ and N₂ sources, which may optionally be in fluid communication with the channel. The apparatus may further include a cleaning solution, which may optionally be in fluid communication with the channel. The compressed gas source may be selected from one or more of the following: air; pentane; CO₂; O₂; or N₂. The compressed gas source may be selected from one or more of the following: air; CO₂; O₂; or N₂. The more than one compressed gas source, may be selected from the following: air; pentane; CO₂; O₂; or N₂. Where pentane is a an analyte of interest, pentane may be excluded as a purging and/or recovery gas.

### BRIEF DESCRIPTION OF THE DRAWINGS

FIGS. 1A-1C show a schematic of a chemo-resistor (MOS gas sensor) and it's bias circuit, where V_{b} is the bias voltage for the sensor and VIₕ is the voltage across the heater (FIG. 1A); (FIG. 1B) shows the equivalent electrical circuit of the sensor in a DC bias; and (FIG. 1C) shows a typical response of a sensor exposed to a certain concentration of a certain gas, wherein 1/Rₐᵢᵣ and i/R_{gas} are the conductances of the sensor in clean air and after exposure to a gas, respectively.

FIGS. 2A-2E show a schematic of a MOS gas sensor and its bias circuit exposed to two different gases in (FIG. 2A); typical transient responses of the sensor to two different gases (⋅ and ▴) are almost the same in (FIG. 2B); a schematic of a MOS gas sensor integrated with a micro-channel and its bias circuit exposed to two different gases in (FIG. 2C); typical transient responses of the microfluidic-based gas sensor to two different gases (⋅ and ▴) are distinct in (FIG. 2D); and a schematic of the gas sensor integrated with a micro-channel is shown in (FIG. 2E), wherein analyte molecules diffuse into the channel, and some of the molecules get adsorbed while some of the adsorbed molecules get desorbed.

FIGS. 3A-3D show a schematic of the experimental setup in (FIG. 3A), wherein the sensor is mounted on a chamber, while three different positions (i.e., FIGS. 3B; 3C; and 3D) are overlaid on a typical normalized transient response of the sensor to a concentration of a gas, with FIG. 3B showing the analyte injection position; FIG. 3C showing the exposure position; and FIG. 3D showing the recovery position.

FIG. 4A shows an exploded view of a schematic diagram of an embodiment of a 3D-printed gas detection apparatus shown in FIG. 4B, having a channel coated with chromium (Cr), gold (Au), and Parylene C, wherein the Cr forms a part of the first layer with Au and the Parylene C forms the second layer of the channel.

FIG. 4B shows a cutaway view of a schematic diagram of the gas detection apparatus shown in FIG. 4A, showing the channel coated with chromium (Cr), gold (Au), and Parylene C, leading from the gas inlet to the gas sensor.

FIG. 5 shows normalized responses from six sensors with six different coating material combinations deposited on the channel to 2000 ppm Ethanol (coatings are as follows: (1) SiO₂ and Parylene C; (2) Parylene C alone; (3) Copper and Parylene C; (4) chromium/gold and Parylene C; (5) chromium and gold; and (6) chromium/gold and Cytonix).

FIGS. 6A-6D show normalized responses for three different analytes (i.e. ethanol (⋅); methanol (▪); and acetone (▴)) with four different channel coatings, as follows: (FIG. 6A) SiO₂ and Parylene C, (FIG. 6B) Cr and Au, (FIG. 6C) Cu and Parylene C, (FIG. 6D) Cr and Au and Parylene C.

FIG. 7 shows typical normalized responses for three different analytes (i.e. ethanol (⋅); methanol (▪); and acetone (▴)), wherein the separation factor is defined to show the differentiation power of the sensor.

FIG. 8 shows a feature space for the sensor with the coating combination of Cr and Au and Parylene C, which had the best performance for three VOCs (Acetone: ∇, Ethanol: ×, and Methanol: ◯) in terms of selectively and recovery time.

FIGS. 9A-9D show normalized responses for three different analytes (i.e. ethanol (⋅); methanol (▪); and acetone (▴)) for four different channel dimensions, as follows: 1=20 mm; d=500 μm (FIG. 9A); 1=30 mm, d=500 μm (FIG. 9B); 1=40 mm, d=500 μm (FIG. 9C); and 1=30 mm, d=200 μm (FIG. 9D).

FIGS. 10A-10F show recorded transient responses for 8 different concentrations (250 ppm-4000 ppm) for 6 different targets, including three alcohols: 2-Pentanol (FIG. 10A), Methanol (FIG. 10B), Ethanol (FIG. 10C), and three ketone: Acetone (FIG. 10D), 2-butanone (FIG. 10E), 2-pentanone (FIG. 10F).

R1 FIG. 11 shows a feature space presentation for all the responses shown in FIG. 10.

FIG. 12 shows a schematic of a breath-analyzer prototype.

FIGS. 13A-13D show normalized responses of the sensor to (FIG. 13A) THC-methanol, and (FIG. 13B) pure methanol at different temperatures, wherein the 3D feature space is shown for (FIG. 13C) THC-methanol and (FIG. 13D pure Methanol, and features F1 and F2 are the points in time at which the normalized response level reaches 5% and 95% of the maximum level, respectively, and F3 is the magnitude of the normalized response at the final read out, wherein (25° C.: ∇, 40° C.: ×, and 80° C.: ◯).

FIGS. 14A-14B show normalized responses for two different analytes A (⋅); and B (▪); the selectivity factor is defined to examine the differentiation power of the sensor is shown in (FIG. 14A) and the sensor response time and selectivity factor between binary mixture of THC-methanol and methanol vs. channel temperature is shown in (FIG. 14B).

FIG. 15 shows a scanning electron micrograph (SEM) to demonstrate pore size of a typical the channel surface coated with parylene C (i.e. pore size is about 50 nm, with a range of between 36 nm and 84 nm).

FIGS. 16A-16B show a typical transient response of the sensor to a concentration of a gas in (FIG. 16A); and the feature extraction method used for identification of the concentration of the analyte is presented in (FIG. 16B), wherein the three selected features are the maximum level of the transient response (F1), the response level at the final readout (F2), and the area under the transient response curve (F3).

FIGS. 17A-17B show the transient response of the sensor to three different concentrations of ethanol, i.e., 1000 ppm (×), 2000 ppm (◯), and 3000 ppm (∇) in (FIG. 17A); and the feature space (using the method described in FIGS. 16A-16B) is presented for identification of the concentration of the analyte in (FIG. 17B).

FIG. 18 shows the regression model used for characterization of the concentration of the analyte (C) with respect to the area underneath the transient response curve (A), wherein the relation between the concentration and the average of the area underneath the curves is linear and each square marker is the average of 5 points and the error bars present the deviation from the average.

FIG. 19A shows a schematic of an embodiment for a pentane detector using a single microfluidic sensor, wherein the sensor uses solenoid valves to expose the sensor to the pentane plume prior to recovery with compressed on-board gas and the purging air exits through the exhaust valve.

FIG. 19B shows a schematic of an embodiment for a UAV-mountable detector for NG leakage monitoring, having a valve network and sensor array ensures rapid sampling of surrounding air for fugitive NG, with compressed air or another source of clean air (uncontaminated with target gases) to recover the sensor.

FIG. 20 shows a schematic of an embodiment for nuisance sewer gas detector including the supporting systems and sensing unit.

FIGS. 21A-21D show two schematic diagrams of multilayer combinations of two fabricated detectors, (FIG. 21A) Detector O (chromium, gold, Parylene C); (FIG. 21B) Detector X (chromium, gold, Parylene C, and Cytonixn); and (FIGS. 21C, 21D) show the contact angle values estimated for DI water on the surface of Detector O and Detector X, respectively.

FIGS. 22A-22D show the normalized transient responses of Detector O (FIG. 22A) and Detector X (FIG. 22C) to 1000 ppm of methanol (X), ethanol (O), i-propanol (⋄), 2-pentanol (□), acetone ( [symbol] ), pentane (⋅), and hexane (+), wherein each experiment is repeated 8 times and the feature space presentation for the seven examined analytes are shown as tested with Detector O (FIG. 22B) and Detector X (FIG. 22D).

FIGS. 23A-23C show the normalized transient responses of (FIG. 23A) Detector O (solid lines) and Detector X (dash lines) to 1000 ppm of methanol, ethanol, 1-propanol, 2-pentanol; (FIG. 23B) the normalized transient responses of Detector O (solid lines) and Detector X (dash lines) to 1000 ppm of acetone, pentane, hexane; and (FIG. 23C) the feature space presentation for Detector O (shown with O markers) and Detector X (shown with X markers) for 7 tested analytes.

FIGS. 24A-24B show an Owen-Wendt determination of the surface free energy of two different channel coating surfaces for (FIG. 24A) Detector O, and (FIG. 24B) Detector X.

FIG. 25 shows the linear relation between the Euclidean distances of the feature vectors of the two Detectors (i.e. X and O) vs. the difference between the surface tension of solid-liquid for the two detectors obtained for different analytes.

### DETAILED DESCRIPTION

Any terms not directly defined herein shall be understood to have the meanings commonly associated with them as understood within the present field of art. Certain terms are discussed below, or elsewhere in the specification, to provide additional guidance to the practitioner in describing the compositions, devices, methods and the like of embodiments, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples in the specification, including examples of terms, is for illustrative purposes only and does not limit the scope and meaning of the embodiments described herein.

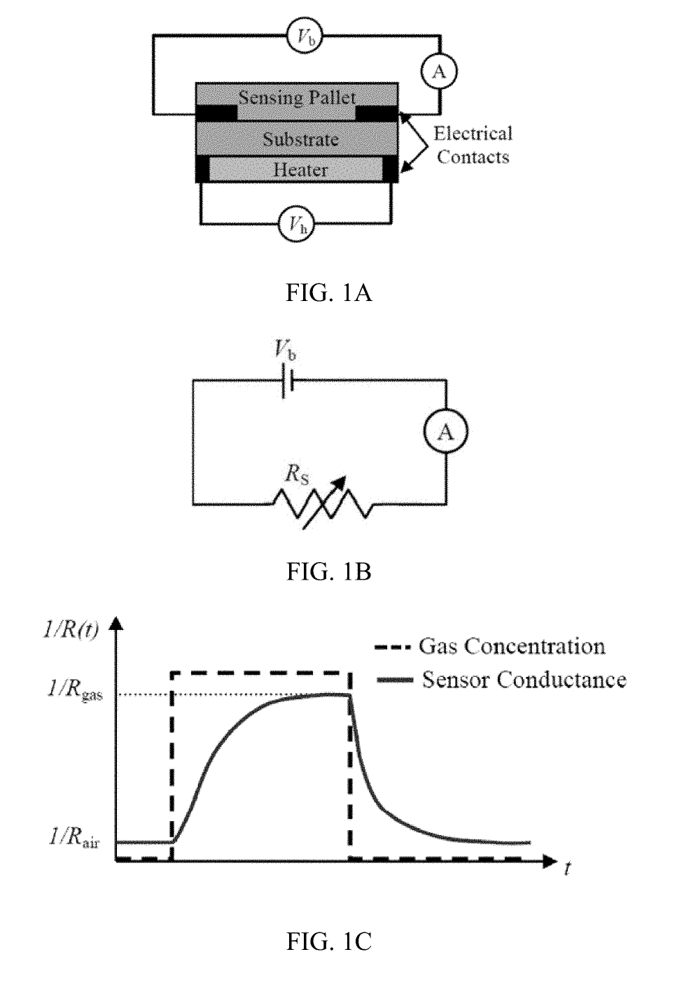

The most widely-used type of gas sensors is Metal Oxide Semiconductor (MOS) gas sensors [23]. In the basic configuration of MOS sensors, which is shown in FIG. 1A, a chemo-resistor is made by deposition of a thick film metal oxide sensing pallet and a thick film thermo-resistor micro-heater on the opposite surfaces of a millimeter-scale ceramic substrate [23].

The electrical behavior of a MOS sensor in a DC bias can be modeled as a variable resistance Rₛ (see FIG. 1B), The value of this resistance depends on the type of the gas molecule, the gas concentration, and the temperature of the sensing pallet. The resistance of the sensor in the clean air is called baseline resistance (Rₐᵢᵣ). The sensitivity (S) of such a sensor is defined by

$$S = \frac{{R}_{air}}{{R}_{gas}} , \qquad ( 1 )$$

where Rₐᵢᵣ and R_{gas} are the resistances of the sensing pallet measured in the clean air and target gas, respectively (see FIG. 1C). The selectivity of a sensor between two gases (i, j) is defined by

$$Sel ( i , j ) = \frac{{s}_{i}}{{s}_{j}} , \qquad ( 2 )$$

where Si and Sj are the sensitivity of the gas sensor to gas i and j, respectively.

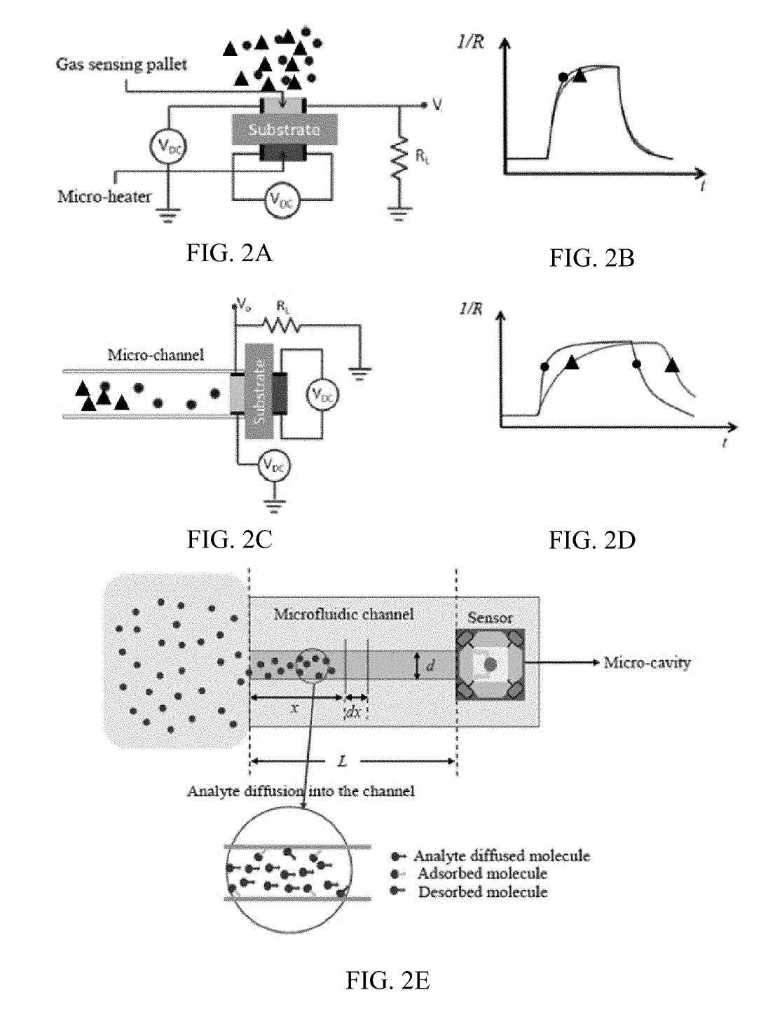

Current off-the-shelf gas sensors are inexpensive and durable, however, they are either made to be evenly sensitive to different gases or fabricated for detecting a single specific target. Hence, differentiating among different gases or gas mixtures using a single sensor is very challenging, as the transient responses of the sensor to two different gases are almost the same. The schematic of a MOS gas sensor and its bias circuit and responses of the sensor to two different gases are depicted in FIGS. 2A and 2B. To enhance the selectivity of the gas sensor, it can be integrated into a microfluidic channel. The schematic of a MOS gas sensor equipped with a channel and its bias circuit is shown in FIG. 2C. The microfluidic-based gas sensor can provide distinct kinetic responses for different gases (see FIG. 2D). The response of such a sensor is dependent on (a) the analyte diffusivity in the surrounding media (air), and (b) the physical adsorption/desorption rate of the gas molecules to/from the channel walls (see FIG. 2E).

The analyte concentration, C(x, t), changes along the channel over time as a result of diffusion of the gas molecules into the channel. The gas concentration can mathematically be predicted by the solving the diffusion—physical adsorption (physisorption) equation [20] of

$$( 1 + \frac{2 {C}_{a}}{d} \frac{α}{( 1 + α C ( x , t ) )} ) \frac{\partial C ( x , t )}{\partial t} = D \frac{{\partial }^{2} C ( x , t )}{\partial {x}^{2}} , \qquad ( 3 )$$

where Cₐ is the number of the surface adsorption sites available per unit volume of the channel, α is a modified Langmuir constant, d is the effective microfluidic channel depth, and D is the analyte diffusion coefficient (diffusivity) in air [24].

As used herein “gas permeability” refers to the rate at which a gas or vapor passes through the channel coating. The gas permeability process includes absorption of the gas or gases into the channel coating and subsequent desorption of the of the gas or gases from the channel coating. The second layer may include a moisture barrier having a gas permeability sufficient to absorb and desorb the gas particles being sampled. Accordingly, the coatings may be optimized for the testing of a particular sample. Factors which may affect permeability of a polymer include the following: chain packing; side group complexity; polarity; crystallinity, orientation; fillers; humidity; and plasticization. Furthermore, the non-reactive metals and non-reactive metalloid compounds used are non-porous and have very low permeability as compared to parylene C, which will stop the gas from going down and reaching to the substrate or the channel and facilitate desorption of the VOC.

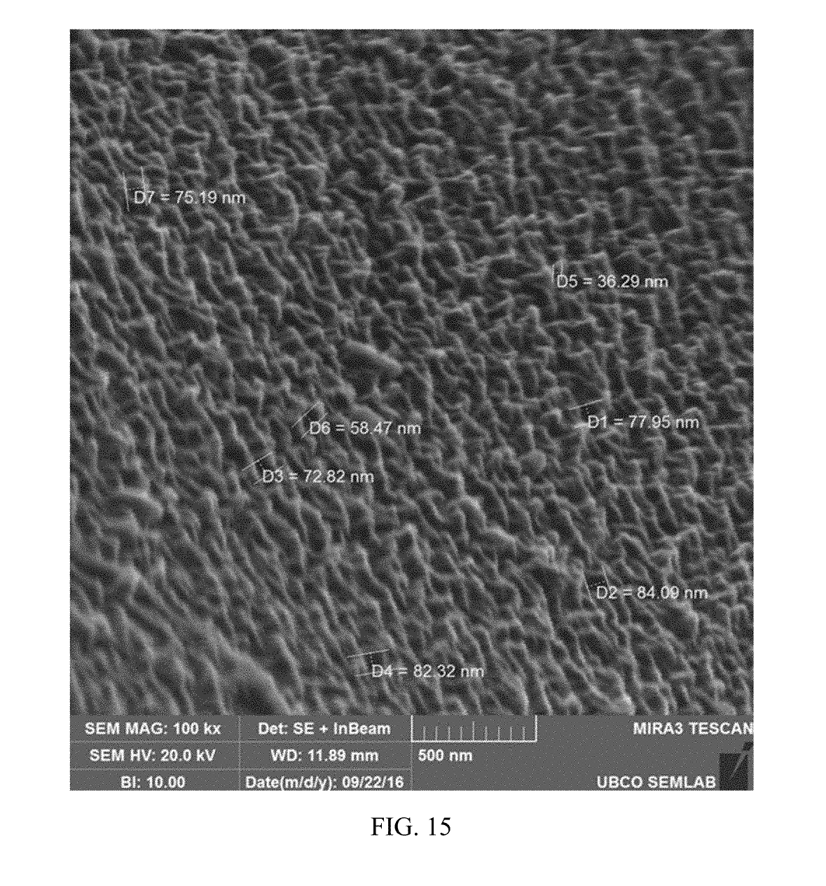

Gas permeability is significant, since sufficient permeability is needed to adsorb and desorb the gas molecules. The molecular dimensions of most VOCs are couple of angstroms so they can diffuse into the voids of Parylene C (which are on average about 50 nm, see FIG. 15) and reach to the first layer of the channel.

TABLE 1
Properties of Parylene N, C and D

| Parylene Barrier | | | |
|---|---|---|---|
| Properties | Parylene N | Parylene C | Parylene D |
| Nitrogen Gas | 7.7 | 0.95 | 4.5 |
| Permeability (cm³- | | | |
| mil/100 in²-24 hr-atm | | | |
| (23° C.)) | | | |
| Oxygen Gas | 30 | 7.1 | 32 |
| Permeability (cm³- | | | |
| mil/100 in²-24 hr-atm | | | |
| (23° C.)) | | | |
| Carbon Dioxide Gas | 214 | 7.7 | 13 |
| Permeability (cm³- | | | |
| mil/100 in²-24 hr-atm | | | |
| (23° C.)) | | | |
| Hydrogen Sulfide Gas | 795 | 13 | 1.45 |
| Permeability (cm³- | | | |
| mil/100 in²-24 hr-atm | | | |
| (23° C.)) | | | |
| Sulphur Dioxide Gas | 1,890 | 11 | 4.75 |
| Permeability (cm³- | | | |
| mil/100 in²-24 hr-atm | | | |
| (23° C.)) | | | |
| Chlorine Gas | 74 | 0.35 | 0.55 |
| Permeability (cm³- | | | |
| mil/100 in²-24 hr-atm | | | |
| (23° C.)) | | | |
| Moisture Vapor | 1.50 | 0.14 | 0.25 |
| Transmission | | | |
| (g-mil/100 in²- | | | |
| 24 hr, 37° C., 90% RH) | | | |

Data from Para Tech Parylene Property Data Sheet and gathered following appropriate ASTM methods

As used herein “substrate” refers to any material suitable for the manufacture of channels or micro-channels and chambers or micro-chambers (for example, the material VeroClear RGD81oTM, polymers, metals, glass, silicon, composite material, plastic or thermoplastic, etc.) and may be chosen based on the coating or coatings being applied to the channels. In most cases the substrates chosen are limited by the coatings being applied and by their ability to be shaped with high resolution. Substrate may be shaped, to provide the desired channel shapes, sizes and dimensions, as well as to provide appropriate sensor locations and associated architectures. The dimensions of the channel or micro-channel may be in the range of 1-10000 μm depth, and may be 1-1000 mm length. The width can be adjusted with the size of the sensor and based on the particular use.

As used herein “coating” refers to any material applied to the surface of the substrate to provide the desired gas permeability and desired diffusion characteristics to facilitate efficient analyte detection. A coating may be comprised or one or more layers and may comprise a first layer having a non-reactive metal or non-reactive metalloid compound and a second layer comprising a moisture barrier, Wherein a second layer is present the moisture barrier may have a gas permeability sufficient to absorb the gas particles being sampled. The non-reactive metal may be selected from one or more of the following: copper; chromium; ruthenium; rhodium; palladium; gold; silver; osmium; iridium; platinum; titanium; niobium; tantalum; bismuth; tungsten; tin; nickel; cobalt; manganese; and zinc, The non-reactive metal may be selected from one or more of the following: copper; chromium; ruthenium; rhodium; palladium; gold; silver; iridium; platinum; titanium; niobium; and tantalum. Alternatively, the non-reactive metal may be a metalloid compound. The metalloid compound may be SiO₂. The moisture barrier with high porosity may be a Parylene or a Polydimethylsiloxane (PDMS). The Parylene may be selected from Parylene C, Parylene N or Parylene D. The Parylene may be Parylene C.

Alternatively, the coating may alter the polarity of the channel. Such polarity altering coatings may be either hydrophobic (for example, Cytonix™ or Teflon™ (i.e. Polytetrafluoroethylene (PTFE); Perfluoroalkoxy alkane (PFA); or Fluorinated ethylene propylene (FEP))) or may be hydrophilic in nature (glass, salts, hydrogels, soap, etc.). As described above the coatings may be arranged in one or more layers and layers may have different properties than one another, depending on the analyte or analytes to be detected. Any hydrophobic and super-hydrophobic material that can be deposited on the surface channel may be used. Additionally, surfaces with synthetic nano-pores or other type of porous coating that can provide more adsorption sites for the particular VOC molecules may be used. The particular coating or coatings chosen may be chosen to provide the desired adsorption or diffusion depending on the intended use (i.e. analyte or analytes being tested, the conditions under which the testing is occurring, the desired sampling time and refresh time, the number and placement of sensors etc.).

The coatings may be added to the substrate in a range of thicknesses depending on the particular use (i.e. on the analyte or analytes to be detected). In some cases, for example when Parylene C is used, the hydrophobicity of the surface can be adjusted with the thickness of the channel coating, so depending on the application one can adjust the thickness and thus the hydrophobicity of the channel coating. The thickness of the coating may be as low as 1 nm with no particular upper limit, but may be limited by the depth of the channel.

As used herein “VOC” or “volatile organic compound” refers to any analyte comprising an organic compound, which may be found in a gaseous or liquid sample.

As used herein “channel” refers to a course or pathway in which a fluid moves and in which the fluid is given direction. Typically, a channel may be any shape or dimension, may be non-linear, may be linear or a combination thereof and may be open along it's length or closed along it's length, depending on the particular gas detection apparatus design and intended use. Furthermore, multiple channels may be used in conjunction with a single sensing element/gas sensor; multiple gas sensors/sensing elements may be used in a single channel (i.e. either distributed along the length of the channel or collected at a channel's terminus or a combination of both); or multiple gas sensors/sensing elements may be used in conjunction with multiple channels.

As used herein “porosity” refers to the “void fraction” which is a measure of the void or empty spaces in a material, and is calculated as a fraction of the volume of voids over the total volume of the material (i.e. between 0 and 1, or as a percentage between 0 and 100%). The porosity may be measured with a BET (Brunauer-Emmett-Teller) measurement device or other surface analysis device. As used herein “porosity” may be a measure of the “accessible void” (i.e. the total amount of void space accessible from the surface) or “total void” as known in the art. Accordingly, “porosity” may be used as an alternative measure for determining the suitability of a particular coating to make up the second layer which includes a moisture barrier.

As used herein “moisture barrier” refers to a water impermeable material or compound. In some embodiments, a parylene (i.e. poly(p-xylylene) polymers) may be used to form the moisture barrier, in part because the parylene polmers may be added in a thin uniform layer that is chemically inert. Some common gas permeabilities and moisture vapor transmission for Paylenes N, C and D are given in TABLE 1. There are a number of parylenes commonly used.

Parylene N

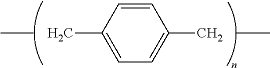

Parylene N has the highest dielectric strength of the three versions, and a dielectric constant value independent of frequency. It is able to penetrate crevices more effectively than the other two versions because of the higher level of molecular activity that occurs during deposition. Parylene N is commonly used in high frequency applications because of its low dissipation factor and dielectric constant values.

Parylene C

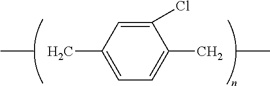

Parylene C differs chemically, having a chlorine atom on the benzene ring that results in a useful combinationof electrical and physical properties including particularly low moisture and gas permeability. This version deposits on substrates faster is than Parylene N, with a consequent reduction in crevice penetration activity.

Parylene D

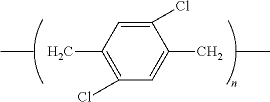

Parylene D has two chlorine atoms added to the benzene ring. This gives the resulting film greater thermal stability than either Parylene N or C, but Prylene D has reduced ability to penetrate crevices as compared to Parylenes N and C.

As used herein “reactivity” refers to the tendency of a substance (i.e. an element or compound) to undergo a chemical reaction, either by itself or with other substances. However, all elements and compounds (except helium) undergo at least some chemical reactions under the proper conditions.

As used herein “non-reactive” refers to a reduced or limited tendency of a substance (i.e. an element or compound) to undergo a chemical reaction, either by itself or with other substances and not a complete absence of reactivity. Furthermore, a non-reactive element or compound will still undergo physical reactions (adsorption and desorption) with the VOCs diffusing through the channel.

A non-reactive metal may be selected from one or more of the following: copper; chromium; ruthenium; rhodium; palladium; gold; silver; osmium; iridium; platinum; titanium; niobium; tantalum; bismuth; tungsten; tin; nickel; cobalt; manganese; and zinc. The non-reactive metalloid compound may be SiO₂.

Applications which require continued monitoring and real time detection such as leakage detection from pipeline and infrastructure, breath analyzers, indoor air quality monitoring devices and etc. may benefit from reduced sampling time.

Methods and Materials

Gas Detector Setup

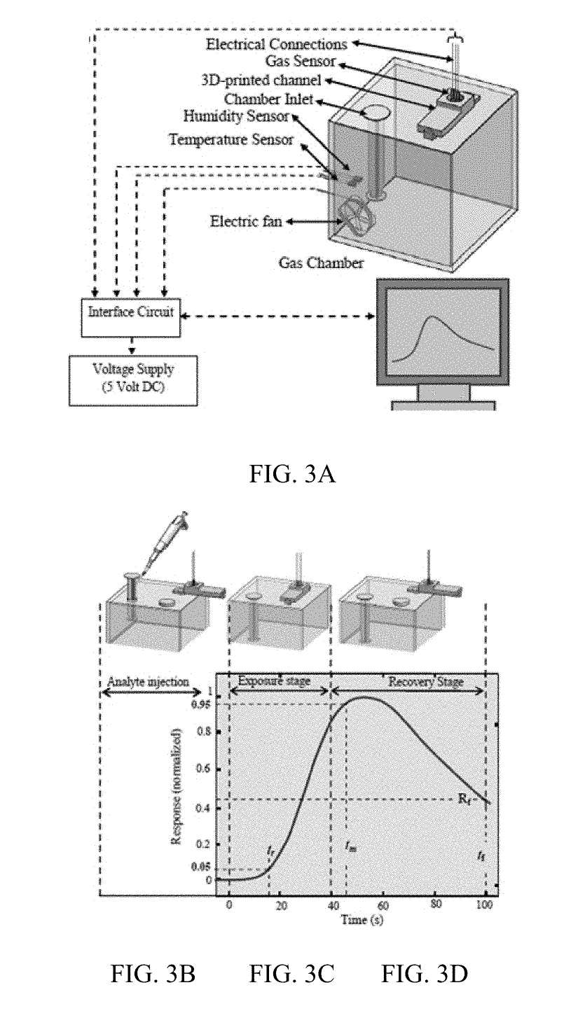

The schematic diagram of the experimental setup is shown in FIG. 3A. The device consists of a gas chamber, three-dimensional (3D) printed microfluidic channel and gas sensor. The sample in liquid phase is injected into the chamber through its opening using a precise Pipet-Lite XLS™ microsampler (analyte injection stage shown in FIG. 3B). After a few minutes, the sample is evaporated into the chamber. The sensor is rotated around the hinge and exposed to the gas inside the 1 L polymethyl methacrylate (PMMA) chamber for 40 seconds (exposure stage shown in FIG. 3C). The gas molecules diffuse into the micro-channel and reach the sensing pallet of the sensor, which is placed at the other end of the channel. The competition between the diffusion process and adsorption of the gas molecules to the available adsorption sites on the channel walls creates a unique response of the sensor (also known as the smell-print). The different smell-prints of gases result in selective sensing of different gases. Finally, the sensor is rotated back to its original position where it is exposed to clean air again and the gas molecules diffuse out from the channel (recovery stage shown in FIG. 3D). Alternatively, the channels could be flushed with clean air or gas (for example, O₂ or CO₂) to shorten the recovery time. The data may be collected (using a microprocessor) for 100 seconds. The device remains in this position for 150 seconds or less where the channel is flushed before the sensor becomes fully recovered and ready for the next test. Most of the experiments were all carried out at the room temperature (25±1° C.), and relative humidity of 40±5%.

Feature Extraction

The typical normalized response of the sensor to a typical gas concentration is shown in FIGS. 3A-3D. Note that the normalization process eliminates the effects of the analyte concentration and baseline variations from the responses. Using equation (2), the sensor conductance (G(t)=1/R(t)) change is normalized as

$${G}_{n} ( t ) = \frac{G ( t ) - min ( G ( t ) )}{max ( G ( t ) ) - min ( G ( t ) )} , \qquad ( 4 )$$

where Gn(t), min(G(t)) and max(G(t)) are the normalized conductance, minimum value of the measured conductance and the maximum value of the measured conductance, respectively. Three significant features are extracted and used from each response [20]: a) tr which is the time at which the normalized response level reaches 0.05, b) tm which is the time at which the normalized response level reaches 0.95, and c) Rf which is the magnitude of the normalized response at the final read out. A 3D feature space coordinate is defined based on tr, tm, and Rf, where each response is depicted as a point (tr, tm, Rf). The regular atmosphere of the laboratory is the background media for all the experiments.

Fabrication Process

The fabrication process for each component of the system is explained below: Gas sensor: A commercially available tin oxide-based chemoresistive gas sensor (SP3-AQ2, FIS Inc.™, Japan) was used in this study. The nominal operating temperature is 300° C. was maintained by applying 5 V DC to the microheater. The bias circuit for the sensor is depicted in FIGS. 1A-1C.

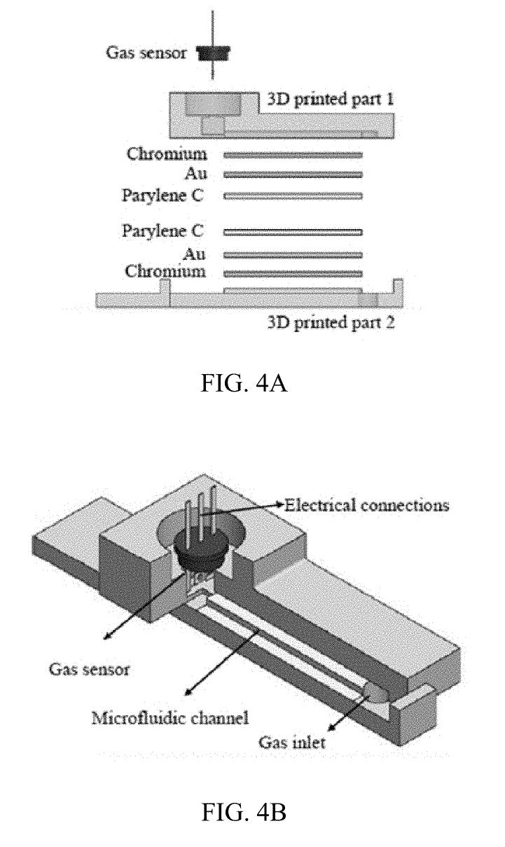

Microchannel: The microchannels/microfluidic channel/channel and micro-chambers/chamber were printed with a 3D-printer (Connex™500), using the material VeroClear RGD810™ (see FIGS. 4A and 4B). To study the effect of channel dimensions and channel surface treatment on the selectivity and recovery time of the sensors, different devices were printed with different channel sizes. Channels with six different dimensions including three lengths (2 cm, 3 cm, and 4 cm) and two heights (200 mm and 500 μm) were fabricated. The width of the channel, which was limited to the dimensions of the sensor chamber, was kept at 3 mm for different channel dimensions.

Channel Coating: The inner surfaces of the micro-channels were coated with single layers and multi-layer combinations of different materials including: gold (with chromium under for adhesion), copper, Cytonix™ (Cytonix LLC™, Product: PFCM 1104V), and Parylene C (poly (p-xylylene) polymer, CAS No: 28804-46-8). The total number of 11 sensors (listed in TABLE 2) were fabricated using different material combinations for the channel coating. For some of the targets (such as Au, Cr, Cu, and SiO₂) the channel surfaces were coated using Physical Vapor Deposition (PVD) sputtering machine (Angstrom Engineering™, Nexdep™ deposition system). Parylene C was coated using a Chemical Vapor Deposition (CVD) Parylene C coating machine (SCS™. PDS 2010 Labcoater™), and for the Cytonix™ the dip in and spin coating methods were both used. Inner surfaces of the microchannel shown in FIGS. 4A and 4B were coated with multi-layer materials including 65 nm gold (with 35 nm chromium under for adhesion) and 4 μm Parylene C.

TABLE 2
Different Channel Coating Used for Sensor Fabrication

| | Single Layer/ | |
|---|---|---|
| Number | Multilayer Coating | Coating Method |
| 1 | VeroClear RGD810 | No coating (3D printed material) |
| 2 | Copper (Cu) | Sputtering |
| 3 | Chromium (Cr) & | Sputtering |
| | Gold (Au) | |
| 4 | Parylene C | CVD |
| 5 | SiO2 | Sputtering |
| 6 | Cytonix | Spin Coating |
| 7 | Cu & Cytonix | Sputtering (Cu) & Spin coating |
| | | (Cytonix) |
| 8 | Cr &Au & Cytonix | Sputtering (Cr and Au) & Spin coating |
| | | (Cytonix) |
| 9 | Cu & Parylene C | Sputtering (Cu) & CVD (Parylene C) |
| 10 | Cr & Au & Parylene C | Sputtering (Cr and Au) & CVD |
| | | (Parylene C) |
| 11 | SiO2 & Parylene C | Sputtering (SiO2) & CVD (Parylene C) |
| 12 | Cr/Au & Parylene C/ | [38] |
| | Cytonix | |

Chamber: A small opening on the chamber (made of PMMA), was provided for both analyte injection and purging clean air into the chamber. An electric fan (DC Brushess. DC24V. 1.41 A. Delta Electronics™), was installed in the chamber to make a uniform environment inside the gas chamber. The microchannel, was attached to the chamber using a screw hinge, which allows the device to rotate on the chamber. The sensor, was first exposed to the clean air.

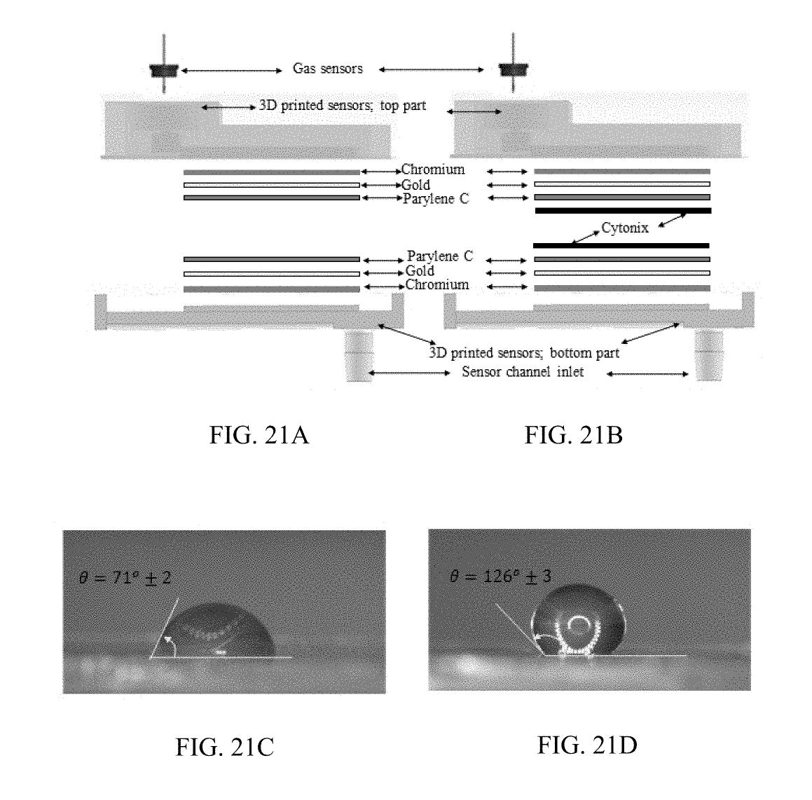

Microchannel: The details of the fabrication process, are described herein and in [38]. In essence, the microfluidic channel is coated with two different coating combinations (as it is shown in FIGS. 21A-21D): (FIG. 21A) Detector O includes three layers of chromium (35 nm), gold (65 nm), and parylene C (4 μm); and (FIG. 21B) Detector X includes four layers of chromium (35 nm), gold (65 nm), and parylene C (4 μm), and Cytonix (100 nm). The dimensions of the channel were kept the same in both detectors: l=20 mm, w=3 mm, d=500 μm, where l, w and d represent the channel length, width and depth, respectively.

Channel hydrophobicity: To show the level of hydrophobicity of the channel surface, the contact angles of a droplet of deionized water (DI water) on both fabricated channel surfaces are estimated (see examples presented in FIGS. 21C and 21D). Each contact angle is measured five times (using ImageJ) and the average and standard deviation. Different surface treatments (resulting in different wettability) may be attributed to the polarity of the top layer coated on the channel [36].

Gas sensor: Gas detectors may consist of 3D-printed parts and a metal oxide semiconductor (MOS) gas sensor (FIGARO, TGS 2602) (see FIGS. 3A-3D and 21A-21D). The detectors, may be connected to sampling chamber or lab environment via the three-way valves.

Analytes: Some of the experiments, were performed using a number of VOCs with different polarities including: alkanes, ketones, and alcohols (which are mentioned from minimum to maximum polarity from left to right). A constant concentration (1000 ppm) of each of the analytes is injected into the system (for different experiments) using a precise micro-sampler (Pipet-Lite XLS). The concentration of the analyte, is kept constant during all the experiments to eliminate the effect of the change in the analyte concentration on the detector response curves.

TABLE 3 lists the properties of the analytes tested here [37]. All the properties are related to each other. For example, as the hydro-carbon chain becomes larger in alcohols the molar mass increases, and on the other hand, diffusion coefficient and vapor pressure both decrease. Also, the larger the hydro-carbon chain the lower the polarity of the compound. This will result in having a smaller relative polarity number and larger boiling point. Similar trends are also seen among the ketone and alkanes.

TABLE 3
List of Analytes tested here with their physical and chemical properties [37].

| | | | | VAPOR | | | | |
|---|---|---|---|---|---|---|---|---|
| | | Molar | DIFFUSION | PRESSURE | | Boiling | γ_{LV}^{p} | γ_{LV}^{d} |
| | | Mass | COEFFICIENT | (20° C.) | RELATIVE | point | AT 20° C. | AT 20° C. |
| GAS | FORMULA | [G/MOL] | [CM²/S] | [MMHG] | POLARITY | [° C.] | IN MN/M | IN MN/M |
| METHANOL | CH₃OH | 32.04 | 0.1520 | 97.66 | 0.762 | 64.6 | 7 | 16.7 |
| ETHANOL | C₂H₅OH | 46.07 | 0.1181 | 44.62 | 0.654 | 78.5 | 4.6 | 17.5 |
| 1-PROPANOL | C₃H₇OH | 60.1 | 0.0993 | 21.00 | 0.617 | 97.0 | 2.9 | 20.8 |
| 2-PENTANOL | C₅H₁₁OH | 88.15 | 0.071 | 6.03 | 0.488 | 119.0 | — | — |
| ACETONE | C₃H₆O | 58.08 | 0.1049 | 180.01 | 0.355 | 56.2 | 3.1 | 22.1 |
| PENTANE | C₅H₁₂ | 72.15 | 0.0856 | 429.78 | 0.009 | 36.1 | 0 | 16.2 |
| HEXANE | C₆H₁₄ | 86.18 | 0.0732 | 120.00 | 0.009 | 69.0 | 0 | 18.4 |

After six minutes, the sample is completely evaporated and unifon ily spread into the chamber. The two detectors are then exposed (using the three-way valves) to the exposure chamber for 40 sec. The gas molecules start diffusing into the dead-end channels through the valves and reach the sensing pallets of the two sensors, which are placed at the other end of the channels. Finally, the detectors are connected to their original positions where they are exposed to the clean air again and the gas molecules diffuse out from the channels (i.e., referred to as the recovery stage). The kinetic responses of the gas diffusion along the channels are recorded (using an Arduino microcontroller) till t=150 sec. This is long enough for the sensor to be recovered). The two detectors remain in this position for a few minutes before the sensors become fully recovered and ready for the next experiment. The experiments are all carried out at the room temperature of 25±1° C. and relative humidity of 30±5%. These conditions are kept constant during the experiments.

The methods and materials described above were employed with respect to the EXAMPLES described herein.

### EXAMPLES

### Example 1

### Channel Coating

The analyte diffusion process was independent of the channel coating material and dependent on the analyte type. However, the adsorption and desorption processes are dependent on both gas type and the channel surface material. Therefore, it was expected that the surface treatment of the channel would results in different transient response profiles. To study the effects of channel coating on the sensor response, a set of materials, as listed in TABLE 2, were tested.

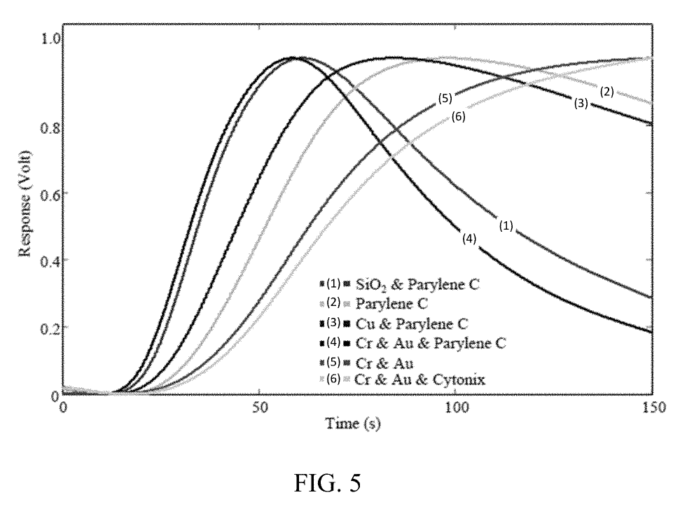

Normalized transient responses of six of the sensors (coatings number 3-4 and 8-11) to 2000 ppm ethanol are shown in FIG. 5. The rest of the channel coatings (coatings number 1-2 and 5-7) did not show significant responses as some of the materials hindered the diffuse-in process. As a result, these five coatings seemed to trap all the ethanol molecules stopping them from travelling along the channel and approaching the sensor. As it can be seen in FIG. 5, the interaction of the gas molecules with different materials was different resulting in varying normalized responses.

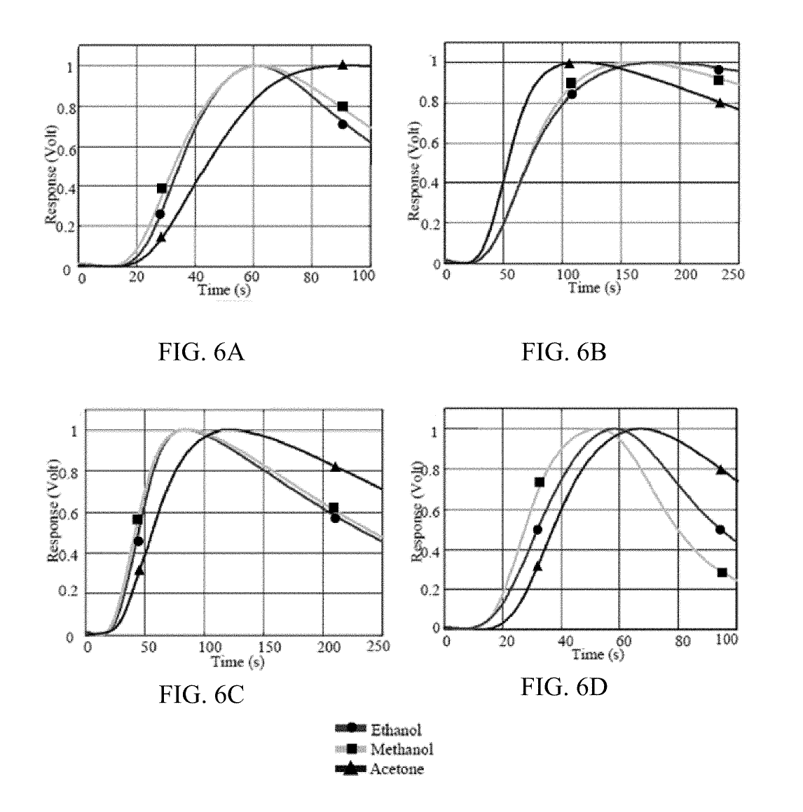

Single metal layer coatings: Among all the channels coated and tested with a single metal layers, gold (with chromium underlayer and parylene C second layer, showed the best response (FIGS. 5 and 6A-6D), as it is one of the most non-reactive materials in nature and was used here to decrease the chemical cross contamination of the gas molecules to the channel walls which eventually results in faster sensor recovery. The chromium layer was coated to increase the adhesion of the substrate to gold. Similarly, a SiO₂ first layer with a parylene C second layer showed a good response (volts) and recovery curve (FIG. 5). However, the Cr and Au coated channel without parylene C also showed a reasonable ability to distinguish ethanol, methanol and acetone (FIG. 5).

First layer (Bottom layer i.e. closest to the channel surface): In case of channels with multilayer coatings, it is observed that the channels coated with different bottom layer materials (even with the same top layer) provide different responses. For instance, the channel coated with three layers of Cr, Au, and Parylene C (with a gold and chromium layer as the bottom coating layers) and Cu and Parylene C (with the copper layer as the bottom coating layer) show different responses to the same concentration of ethanol. This is due to the permeation of the gas molecules through the top layer and reaction with the bottom coating layer. In choosing a first layer, it is preferred in some embodiments that the first layer physically interacts (i.e. non-specifically and reversibly via van der Wahl's forces) with the VOC, but does not chemically interact with the VOC.

Second layer (Top layer i.e. on top of the first layer): The preliminary experiments revealed the importance of the porosity of the top coating layer. In essence, the number of surface adsorption sites available per unit volume of the channel (Ca in equation (3)) is greater in channels with higher porosity. As it is shown in FIG. 5, the diffuse-in and diffuse-out processes of ethanol was more rapid in the channels with the combination of Cr and Au and Parylene C coatings, whereas, the coating combination of Cr and Au and Cytonix shows the slowest response. This suggests that more physical adsorption occurs in the case of Cr and Au and Cytonix channel coating. Thus, Parylene C is a good candidate for the top layer coating material as it can be coated as a thin polymer film, which is chemically inert. It also has high porosity [25], which increases physical adsorption of the gas molecules to the channel walls that eventually increases selectivity of the sensor. In addition, Parylene C provides a pinhole free coating and a lower permeability (as compared to other similar polymers) and has been recently used in the development of GC columns [26] as well as a material for moisture barrier in numerous applications [27]. The latter is potentially significant for gas sensing, since the gas sensors are subject to errors as they are vulnerable to ambient fluctuations such as humidity and temperature change. Therefore, the response of a sensor depends on not only the analyte concentration, but also the ambient conditions (particularly humidity). Therefore, in high precision sensing applications, such as breath analyzers, fluctuation in humidity [28] may result in false signals. Thus, the use of an effective moisture barrier such as Parylene C along the channel may reduce the error caused by humidity. In choosing a second layer, it is preferred in some embodiments that the second layer if used has porosity so that the VOC has access to the first layer and is also chemically inert. Furthermore, it may also be useful for the second layer to have moisture barrier properties.

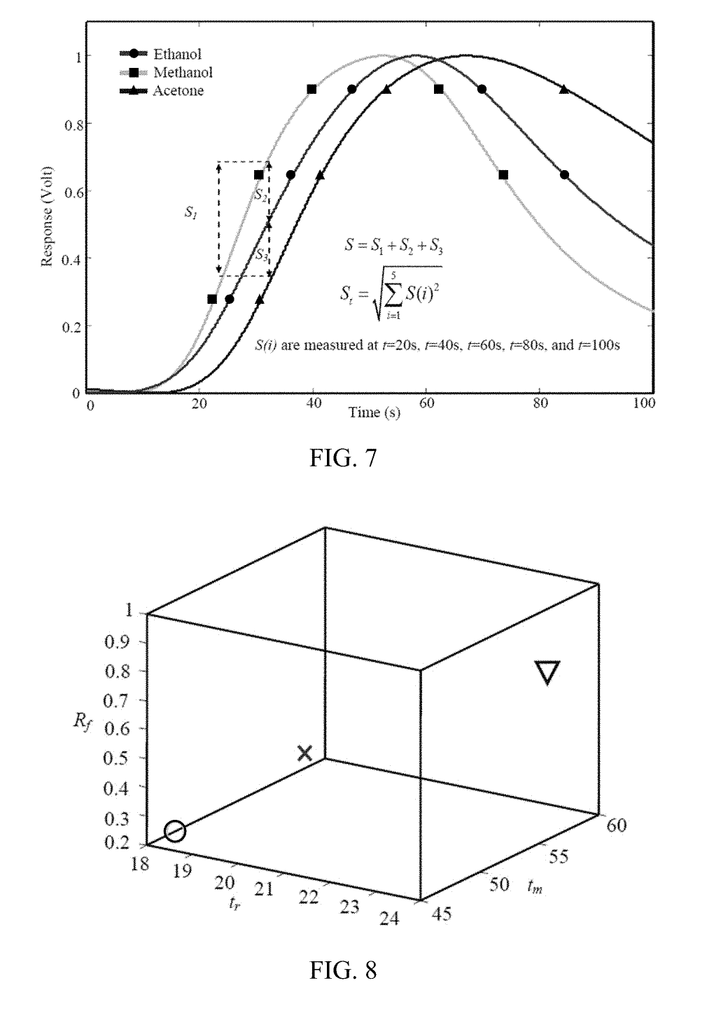

Analytes: Three different analytes including ethanol, methanol, and acetone were tested to compare the selectivity of the fabricated sensors among different gases. These gases were selected to show the capability of the device in differentiating alcohol and ketone vapors. Four out of the eleven fabricated sensors showed acceptable selectivity among the three selected analytes. The temporal responses obtained from the device are normalized to fit within the magnitude range of [0, 1], eliminating the influence of the analyte concentration on the shape of the responses. Normalized responses for each of the sensors to 2000 ppm of each of the three analytes are depicted in FIGS. 6A-6D. As it can be seen in FIGS. 6A-6D, each of the four sensors give unique responses corresponding to different tested analytes such that the finger-prints of three analytes on each of the four selected sensors were distinct. However, different sensors may distinguish these three analytes differently. In other words, it may be observed that from one sensor to another the level of segregation between analytes may be different, showing different selectivity among the sensors tested. A better quantitative comparison may be evaluated based on calculating indicators of selectivity and the recovery time of the sensor to find the optimum material for the treatment of the channel of the proposed gas detector. For instance, FIG. 7 shows typical responses of one of the sensors against three different analytes. A selectivity factor is defined as S=S1+S2+S3, in which S1, S2, and S3 are the absolute values of the distances between the amplitude of responses of methanol-acetone, ethanol-methanol, and acetone-ethanol, respectively, at five different time points (t=20 s, t=40 s, t=60 s, t=80 s, and t=100 s). The square root of the sum of square of the selectivity factors at five points is used as a measure of selectivity of different sensors. Another factor for determining the sensor performance is the recovery time: in essence, the sensor with the lower recovery time is preferable.

Optimization of coating: The selectivity and recovery time of the fabricated sensors are all compared and listed in TABLE 4. In this table, the sensors are listed based on two major categories: coating materials and dimensions. The average pick time of each sensor, which is the mean of three time points for which the sensors have the maximum readout for three different analytes, were also calculated and listed. It is observed that the smaller the pick time value the faster the recovery of the sensor. The average pick time was used to rank (in the order of 1 to 4, from the lowest average pick time to the highest, respectively), and hence compare the speed of the recovery of different detectors. The sensors were also ranked based on their selectivity factor (as explained above). The effect of both coating materials and channel dimensions are separately investigated through the above ranking schemes. The results show that the Cr and Au and Parylene C coated sensor provides the maximum selectivity and the minimum recovery time among all the coating materials tested here. This means that the proposed coating combination decreases the cross contamination and the chemical adsorption and increases the physical adsorption (and hence selectivity). To perform a quantitative comparison of the response of the sensor to different analytes three features (tr, tm, Rf) are extracted from each normalized response. The feature space for the sensor with the coating combination of Cr and Au and Parylene C, which shows the best performance in terms of selectively and recovery time, is shown in FIG. 8. It will be appreciated by a person of skill that the optimum coating will depend on the VOCs being tested.

TABLE 4
Comparison of the Separation Factor and Recovery
Time Among the Fabricated Sensors

| | | Channel | Channel | Average | Peak | | |
|---|---|---|---|---|---|---|---|
| | | Length | Depth | Peak Time | Time | Selectivity | S |
| | Coating | (l) | (d) | (seconds) | Rank | Factor (S) | Rank |
| Sensors with | Cr—Au- | 30 mm | 500 μm | 59.37 | 1 | 1.49 | 1 |
| Different | Parylene C | | | | | | |
| Coatings | Cr—Au | | | 154.07 | 4 | 1.07 | 3 |
| | SiO₂- | | | 72.25 | 2 | 1.08 | 2 |
| | Parylene C | | | | | | |
| | Cu- | | | 98.51 | 3 | 1.01 | 4 |
| | Parylene C | | | | | | |
| Sensors with | Cr—Au- | 20 mm | 500 μm | 51.18 | 1 | 1.37 | 4 |
| Different | Parylene C | 30 mm | 500 μm | 59.37 | 2 | 1.49 | 3 |
| Dimensions | | 40 mm | 500 μm | 67.25 | 3 | 1.52 | 2 |
| | | 30 mm | 200 μm | 68.56 | 4 | 1.74 | 1 |

### Example 2

### Channel Dimensions

After choosing the preferred coating combination of the tested coatings listed in TABLES 2 and 4, for the tested VOCs, Cr and Au and Parylene C were preferred. This coating was then tested to study the effect of the channel dimensions on the response of the sensor, sensors with three different channel lengths and two different channel depths are fabricated and tested (see TABLE 4). The ranking procedure explained above was also used to quantify the effect of the channel dimension on the selectivity and recovery time. In general, there is an opposite trend in rankings based on the selectivity and recovery time for sensors with different dimensions as explained below.

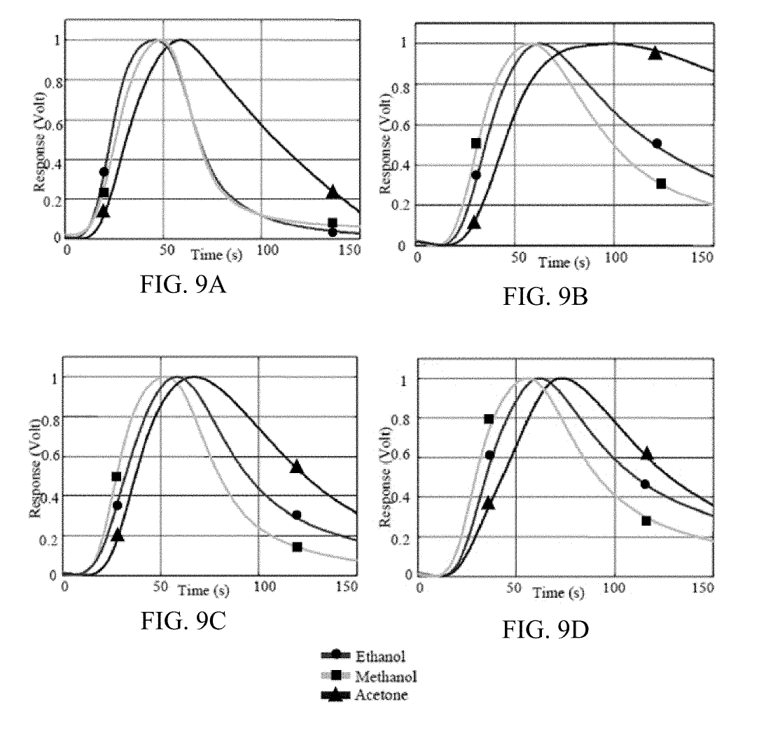

Channel depth: Normalized responses for three different analytes (ethanol, methanol and acetone) for four different channel dimensions: (i) l=20 mm, d=500 μm, (ii) l=30 mm, d=500 μm, (iii) l=40 mm, d=500 μm, and (iv) l=30 mm, d=200 μm (l is the length and d is the depth of the channel) are depicted in FIGS. 9A-9D. As expected, the sensors with higher channel depths are recovered faster. According to equation (3), increasing the depth of the channel decreases the effect of physical adoption, which will result in changing the diffusion-physisorption equation to only diffusion equation for deep channels. In this case (which is only diffusion-dependent), the only analyte related parameter in the equation is D (gas diffusivity). On the other hand, by decreasing the channel depth, the effect of Ca and α in Equation (3) increases and more adsorption and desorption dependency will be observed in the response. Thus, channels with smaller depths are recommended to differentiate gases with similar diffusion coefficients.

Channel length: When examining two gases (with different diffusion coefficients), increasing the length of the channel increases the diffusion time, which results in a larger difference in the temporal responses of the sensor (see FIGS. 9A-9D). In other words, increasing the length of the channel slows down the diffusion process and increases the selectivity of the sensor. However, longer channels result in longer recovery time for the sensor. Therefore, considering the trade-off between the selectivity and the recovery time of the sensor, the preferred dimension of the channel for the VOCs tested and with the tested coatings was l=30 mm, d=200 μm (see TABLE 4).

### Example 3

### Analyte Concentration

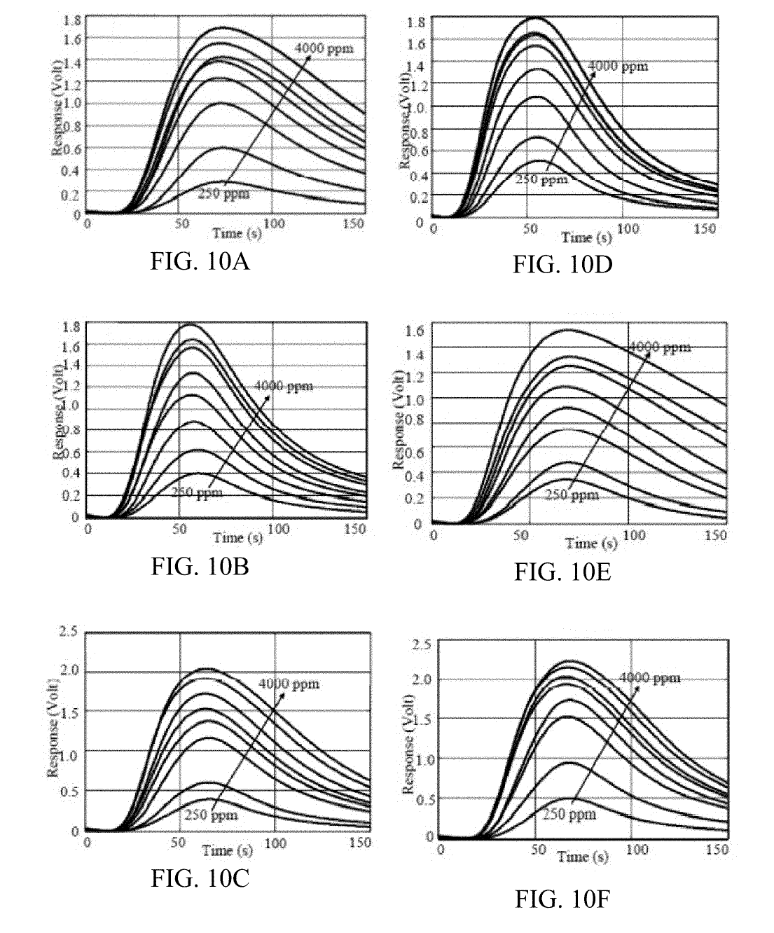

After adjusting the sensor coating and dimensions, the coating of Cr and Au and Parylene C and the dimensions of l=30 mm and d=200 μm are used for verifying the selectivity of the sensor. A wide range of concentration (250-4000 ppm) of 6 different target gases were selected among alcohols (including 2-pentanol, ethanol and methanol) and ketone vapours (including acetone, 2-butanone and 2-pentanone). As recorded transient responses for 8 different concentrations for 6 different targets is shown in FIGS. 10A-10F; the sensor differentiated among different concentration of gases. As presented in FIG. 11, the feature space shows the analytes are successfully separated in the 3D space. The feature vectors of the responses related to each analyte at different concentrations form a clear-cut cluster in the feature space (see FIGURE No mathematical tool was needed for mapping the responses into the feature space, and only one simple feature extraction method [20] was adequate for the determination of the positions of the target analytes in the feature space. The feature space of a particular device was universal and requires hardly any modification when applied to different analytes.

The gas detector operation is humidity and temperature dependent. Ambient temperature and humidity dependence of the responses provided for a specific analyte may be considered as sources of error, which causes displacement of the feature vector related to each analyte in the feature space. This arises from the fact that the analyte diffusion/physisorption along the channel/to the channel walls are both strongly temperature-dependent processes. These errors caused by ambient fluctuations introduce drift-like terms into the responses of the sensor, which causes false measurements. Therefore, the ambient temperature and humidity are controlled during all the experiments. The apparatus may be further optimized to minimize the effect of humidity and temperature fluctuation on the response of the sensor.

Applications based on diffusion may include breath analyzers in which the sample is collected in a chamber and exposed to the sensor. Applications based on flow may also include breath analyzers in which the person blows into the device directly and the flow can be regulated using a flow regulator.

### Example 4

### Detection of Tetrahydrocannabinol (THC)

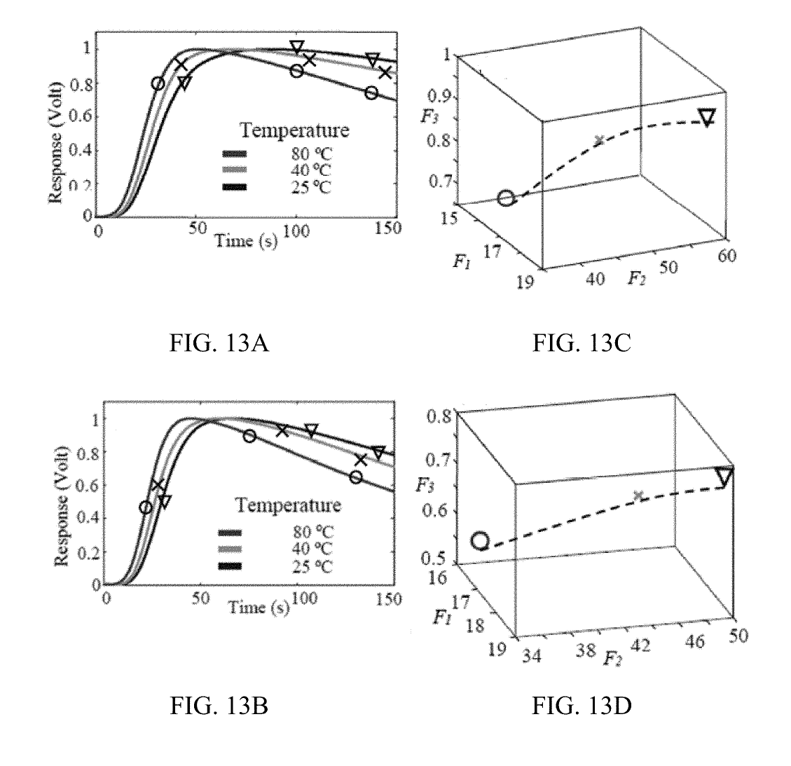

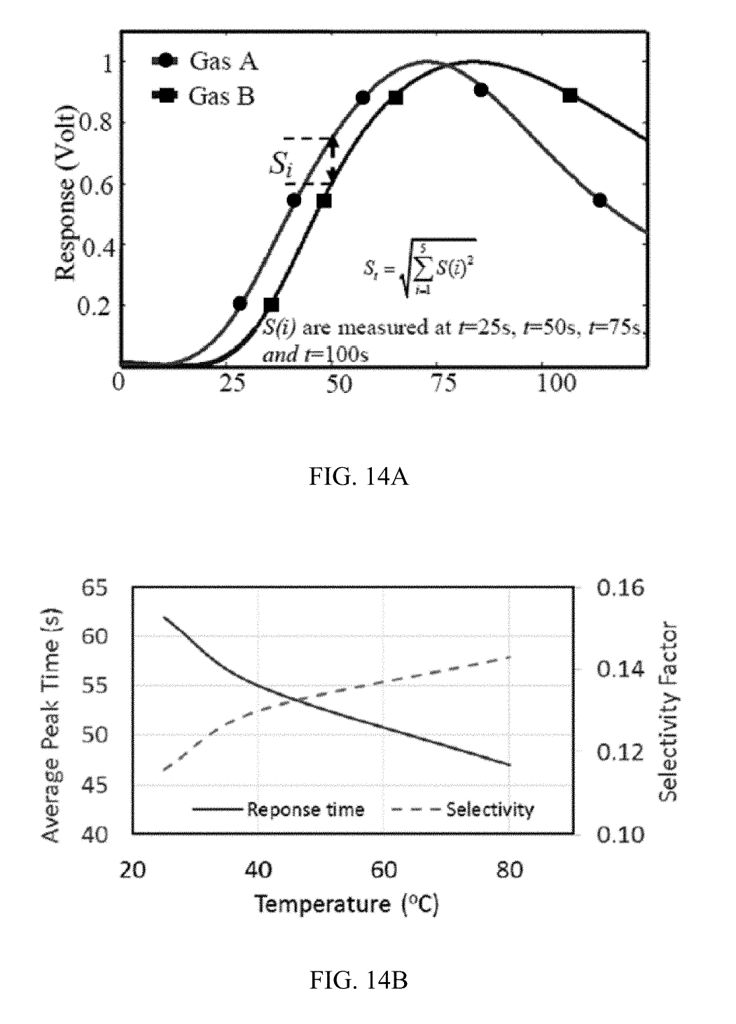

An embodiment of the apparatus was also tested for detection of cannabis in human exhaled breath. The tested embodiment was capable of differentiating small concentrations of Tetrahydrocannabinol (THC) in presence of other volatile organic compounds (VOCs). The main advantage of the proposed device over previous microfluidic-based gas sensors [30-31] is the integration of heaters along the micro-channels to enhance the diffusion rate of the THC molecules in the channel and decreasing the sensor response and recovery time from 15 minutes to below 200 s. Detection of THC in breath has been used as an indicator of cannabis use [32]. However, as there are traces of other VOCs in the breath, it is important to differentiate among different gases, and pinpoint the distinct “smell print” of THC. General purpose Metal Oxide Semiconductor (MOS) gas sensors are sensitive and not selective of different gases [33]. As described above, micro-channels may be integrated with these sensors to enhance their selectively (FIGS. 2A and 2C) [30]. However, these microfluidic gas sensors are not suitable for detection of large molecule gases (such as THC) as the diffusion process is slow and takes more than few minutes [31]. In this example, the sensor response time was decreased by modulating the temperature of the diffusion channel. The sensor assembly was fabricated using a similar method as explained in [30]. To control the temperature of the diffusion channel, a platinum heater wire is integrated along the channel. The response time and selectivity of the sensor for THC-methanol binary mixture (1 mg/mL solution in methanol) and pure methanol were studied at different temperatures (25° C., 40° C. and 80° C.). A method described in [31] was used to characterize the sensor response (FIGS. 13A-13D). The sensor recovery time for THC-methanol mixture at 25° C. was approximately 15 minutes, and as the temperature is increased to 80° C., the recovery time is reduced to under 3 minutes. The slow recovery, which is attributed to high molecular weight of THC, was not observed for pure methanol. Therefore, the overall sensor response time was decreased drastically for THC detection by addition of the heater. Increasing the micro-channel temperature has another important effect: enhancing the selectivity. As can be seen in FIGS. 14A-14B, the selectivity of the device is increased at higher temperatures as bigger molecules of THC in the binary mixture are more actively involved in the diffusion process and react with the sensor. It must be noted that the observed response for the binary mixture of THC-methanol is distinct for each THC concentration, and we have successfully detected THC concentrations as low as 50 ppm. In contrast to previous microfluidic gas sensor designs, the selectivity of the sensor was not compromised when achieving faster response times such that the heater embedded channel design would be suitable for detection of larger molecules including THC. This embodiment may provide a low-cost breath analyzer device, which may provide a powerful tool for roadside testing or also for personal monitoring purposes.

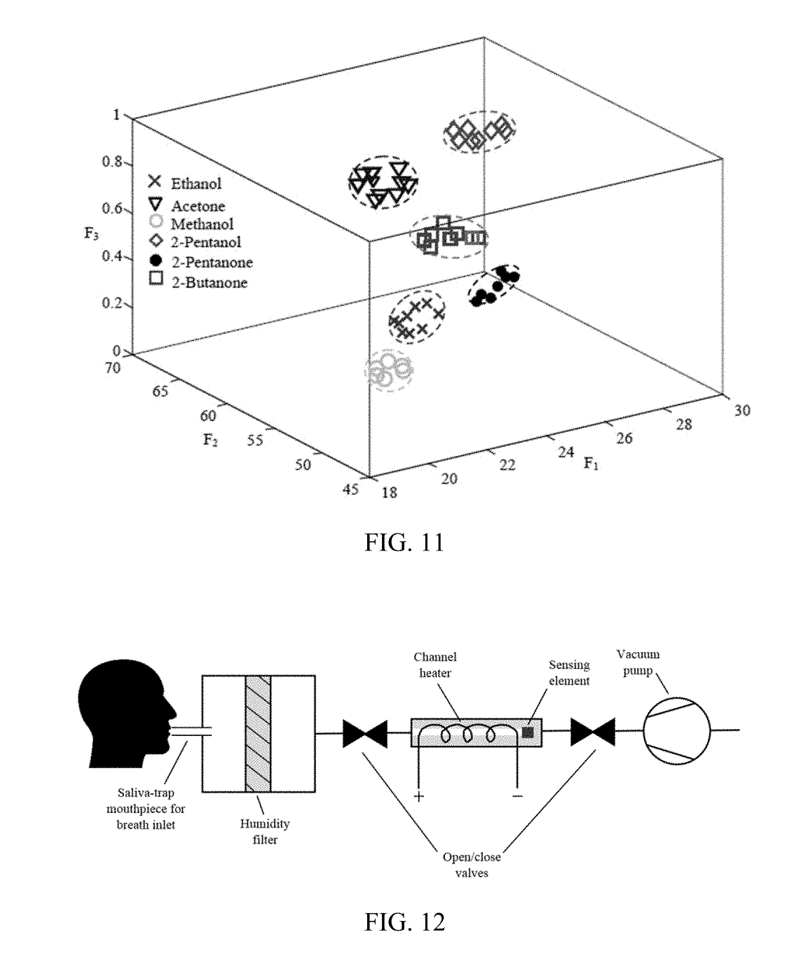

The embodiment shown in FIG. 12, shows one way to control the temperature of the diffusion channel, a heater is shown integrated along the channel. A benefit of the embodiment shown in FIG. 12 is the integration of a heater along the micro-channels to enhance the diffusion rate of the THC molecules in the channel and decreasing the sensor response and recovery time to below 200 S. In contrast, some microfluidic gas sensor designs do not have the selectivity of the sensor in combination with a faster response when used to detect larger molecules, including THC.

The sensor selectivity may be further be enhanced by creating a flow (advection) of gas inside the micro-channels. Also, a water trap is shown in FIG. 12 to minimize large droplets of moisture entering device. The sample enters an antechamber; the force of exhalation drives the sample through a humidity filter and into the sampling chamber. A one way valve can be used ensure gas does not escape through the inlet. A small vacuum pump draws in fresh air from inlet and out the exhaust port to recover the sensor.

A 3D-printed microfluidic platform is fabricated by integrating a chemo-resistor with a channel. Using a novel coating combination, a surface treatment on the inner walls of the microfluidic channel is carried out, which enhances the selectivity power of the device. Different coating materials are tested and compared to choose the best material in terms of giving the maximum selectivity and the minimum sensor recovery time. The geometry of the channel is then optimized after comparison of the results of sensors fabricated with different channel dimensions. Embodiments may be developed as low-cost (˜$10), portable and highly selective gas detectors, which provide a powerful tool for numerous applications including personal monitoring of exhaled breath for patients suffering from different diseases, biological analysis, safety and environmental monitoring, and analytical chemistry.

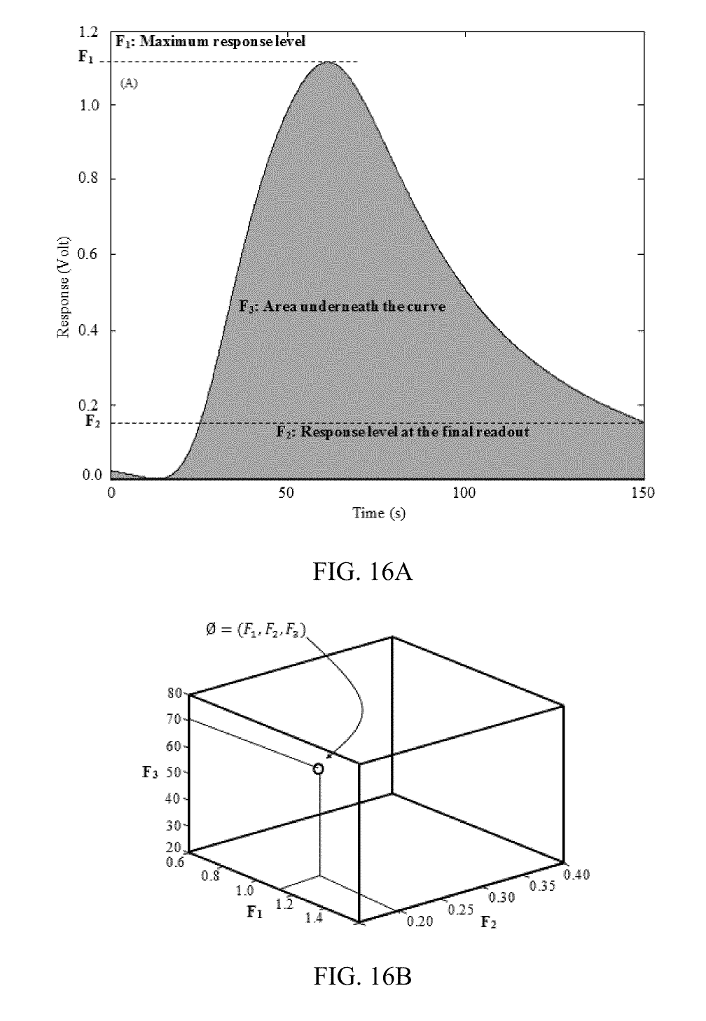

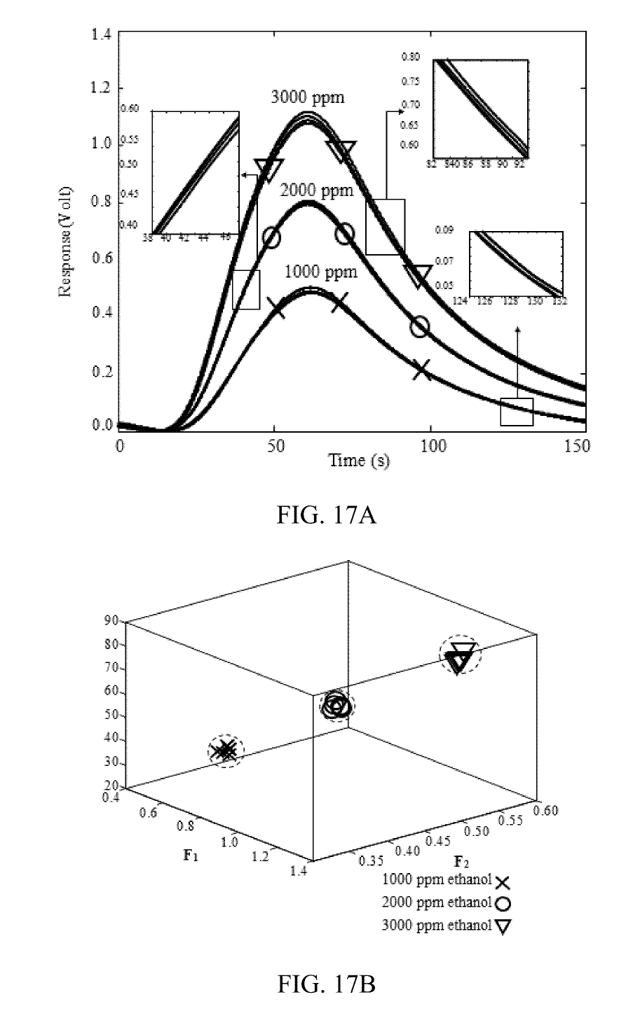

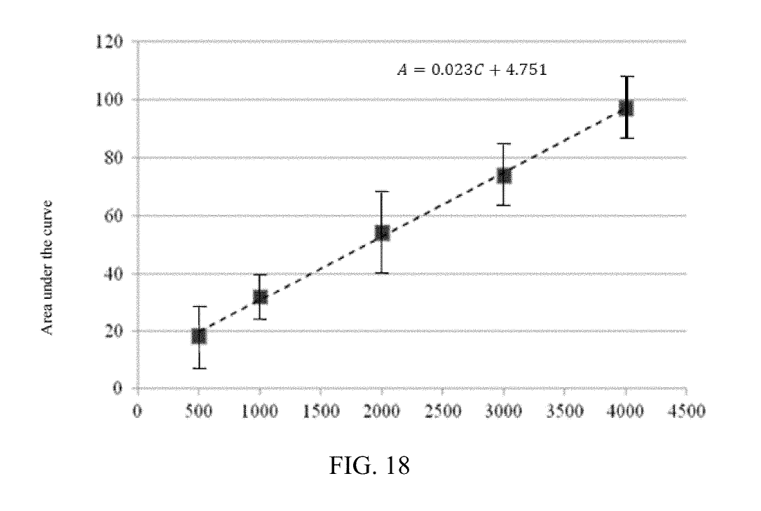

A different method of feature extraction is also used for characterization of the concentration of the analyte. Three different features are extracted from each transient response (see FIG. 16A). The signal maximum response level (F1), the response level for the final readout (F2), and the surface area underneath the response (F3) are the three extracted features from each transient response. The feature vector (ø) extracted from the transient response is shown in a 3D space in FIG. 16B. The transient responses of the sensor with Cr and Au and Parylene C channel coating and dimensions of l=40 mm, w=3 mm, d=500 m are shown in FIG. 17A. The transient responses are shown in FIG. 17A, representing the repeatability of the device for each concentration. Some parts of the transient responses are magnified to show the reproducibility of the response for each concentration. The feature vectors related to each concentration are segregated (see FIG. 17B) in the feature space. The results show three separated spheres, representing the separation capability of the device between different concentrations of the same analyte. A regression model is used to show the linear relation between the concentration and the area underneath the curve (see FIG. 18).

### Example 5

### Natural Gas Leakage Detection

An embodiment of the apparatus was also tested as an automated and reliable means for monitoring of natural gas leakage in pipelines and around pump stations. In particular, a microfluidic-based sensor as described herein may be deployed using an unmanned aerial vehicle (UAV) for timely and precise detection of natural gas leakage at storage sites and along pipelines. Such a device may be operated easily by pipeline maintenance technicians with basic training to remotely inspect natural gas infrastructure including pumps, tanks and pipes wherein the natural gas infrastructure may have limited everyday access. The sensor can be used for detection of methane, ethane and pentane.

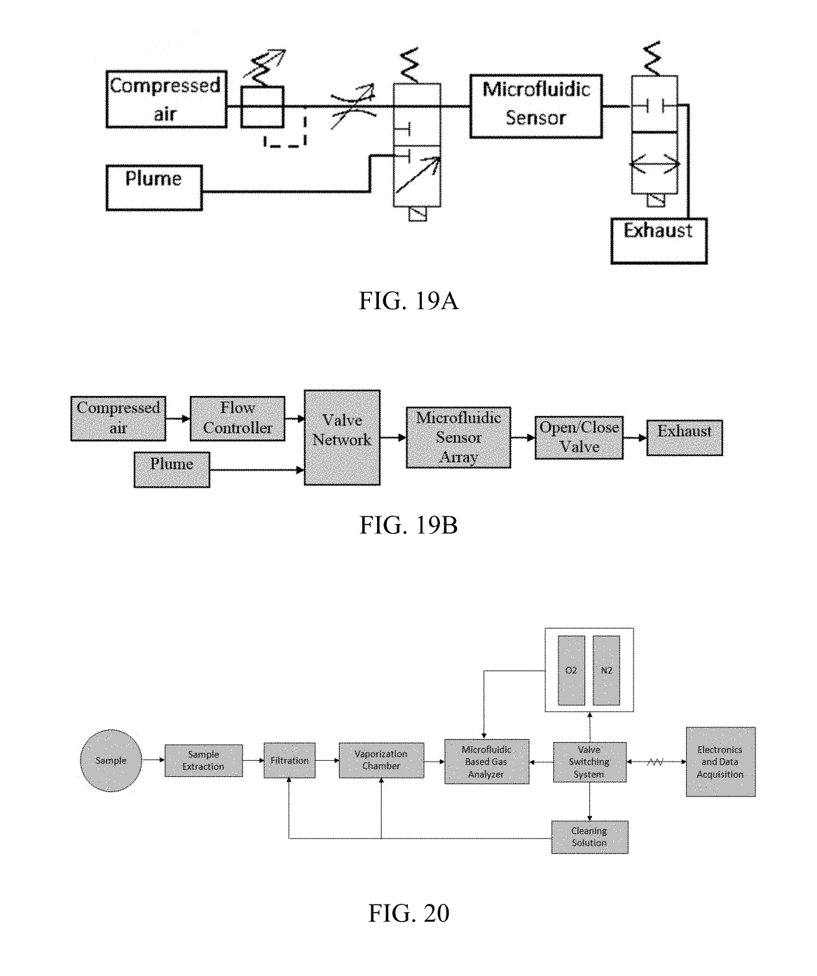

Features of this embodiment may include: a sensor recovery process which is capable of automatically regenerating the saturated sensors using a compressed air recovery chamber and electrically actuated solenoid valves in order to continuously monitor the infrastructure for leakage detection; the slope of the “exposure to pentane”, which is representative of a gas concentration, may be chosen as the main feature of the response, whereby this feature extraction process allows the device to determine the concentration of the desired analyte; the capability to switch between multiple channels for an uninterrupted detection operation wherein there may be a manifold controlled by micro-valves are used; the sensor may be installed in a mobile platform such as a UAV to enable mobile detection of different gases and to achieve this goal a novel sampling procedure was developed to enable sampling consistent amount of gas as the platform is moving; and an onboard microprocessor may be used to relate the UAV flight path to sensor readings of the methane concentration (see FIGS. 19A and 19B).

### Example 6

### Nuisance Sewer Gas Detection

An embodiment of the apparatus is also envisaged, wherein the sensor technology may be used to monitor sewer gases and identify “hotspots” of gas production for targeted treatment. Particularly, the gas sensor may be used for detection of nuisance gases, some of which are odorous or even hazardous. For example, hydrogen sulfide, ammonia, carbon dioxide, methane and nitrous oxide, among other greenhouse gases. The embodiment may be relatively independent and low-maintenance, and may have a streamlined data communications to collect, transmit, analyze and store data to inform users' mitigation strategies in real-time.

Features of this embodiment may include: an aerofoil design is used to minimize the risk of obstruction in the turbid environment, wherein the configuration may be developed to allow the device to be positioned along the side of the pipe to avoid large sediments at the bottom of the pipeline; a shared inlet/outlet channel positioned on the downstream end of the apparatus to avoid blockage due to fast-flowing suspended organics and other waste, which may be combined with a high pressure air source which may be used to purge the previous sample and dislodge any debris build-up and wherein negative pressure may be used to draw the next sample through the inlet; a membrane-less microfiltration mechanism may be used to ensure that the sensing unit is not in contact with microorganisms or debris that can interact with the sample and bias the sensor reading or create nuisance compounds, whereby the microfiltration mechanism is based on the use of inertial microfluidic particle sorters; and since the sensor may use oxygen (O₂) to recover between samples, onboard compressed gas may be used to flush the micro-chamber and channel, whereby the sensor may recover to the baseline, and a neutral gas (N₂) may be used to purge O₂ and any remaining sample from the sensing unit and into the surrounding environment through an exhaust outlet (see FIG. 20).

### Example 7

### Effect of Channel Coating Hydrophobicity and Analyte Polarity on the Gas Detection Capability of a Microfluidic-Based Gas Detector

Transient responses were recorded using the two fabricated detectors (X and O) and a feature extraction method is then applied to the transient responses to compare selectivity of the two detectors using the Euclidean distances of features in the feature space. Following the characterization of channel coating and its polarity for each of the detectors, the interaction between the analyte and the surface of the microchannel was quantified based on the surface free energy of the detector channel surfaces.

Sensor Response and Selectivity.

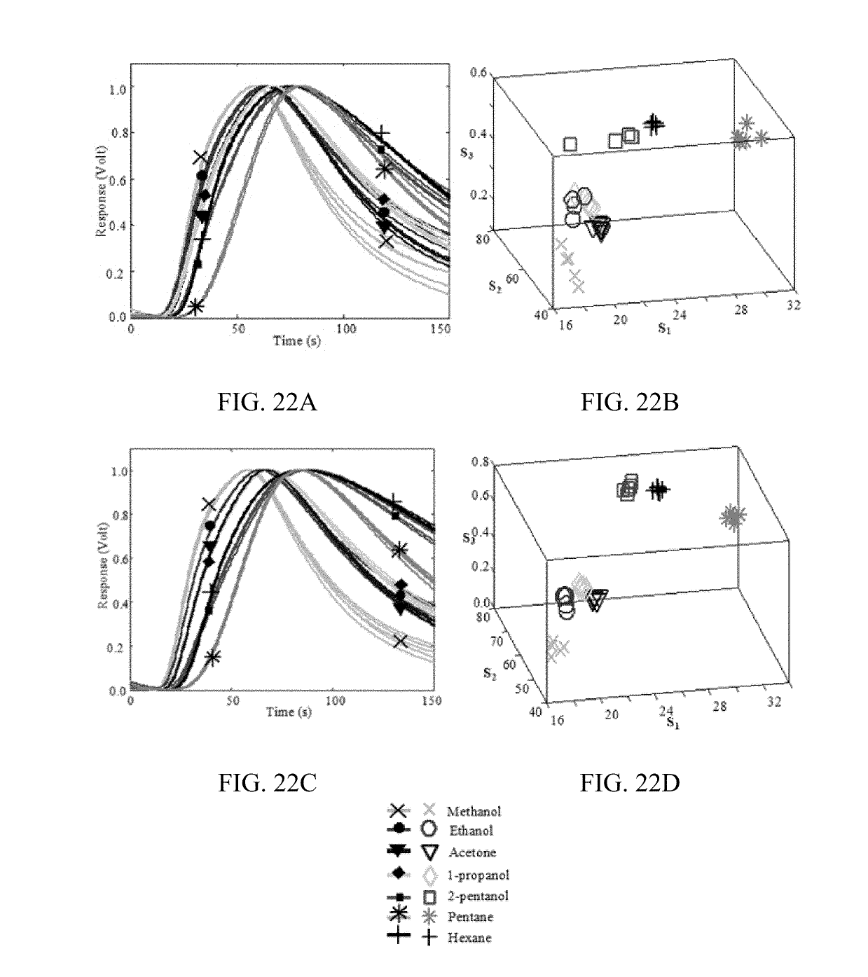

The temporal responses obtained from the sensors are normalized between 0 to 1 (for ease of comparison). The results are shown in FIGS. 22A and 22C for Detectors O and X, respectively. Each experiment was repeated 8 times and for each detector, the response curves show that diffusion-physisorption procedure and accordingly the slopes of the curves during exposure and recovery change as the target gas changes. As seen in FIGS. 22A and 22C, these slopes are steeper for polar gases (e.g. methanol) as compared to non-polar gases (e.g. hexane), Also, The Detector X's normalized responses are more distinct compared to Detector O.

To better visualize the selectivity capability of the detectors, a feature extraction method (as described in [30]) was used to demonstrate the results in a 3D feature space. Three different features are extracted from each normalized transient response: including: 1) S₁: the time at which the normalized response level reaches 0.05; 2) S₂: the time at which the normalized response level reaches 0.95; and 3) S₃: the magnitude of the normalized response at the final read out. The extracted feature vectors obtained from each set of transient responses are shown in FIGS. 22B and 22D for Detectors O and X, respectively. The results shown in FIGS. 22A-22D demonstrate segregated clusters of feature vectors, representing the separation capability of the two detectors among different analytes. It is observed from the feature spaces (FIGS. 22B and 22D) that Detector X (coated with Cytonix) has a better separation capability as the clusters are concentrated with less overlap. To compare quantitatively the selectivity of the two detectors (O and X) among different analytes, the 3D Euclidean distances of the average feature vectors (the mean of each feature component for each analyte) were calculated for each pair of the examined analytes in the feature space using Equation (1):

$$D = \sqrt{{( Avg {S}_{1 i} - Avg {S}_{1 j} )}^{2} + {( Avg {S}_{2 i} - Avg {S}_{2 j} )}^{2} + {( Avg {S}_{3 i} - Avg {S}_{3 j} )}^{2}} \qquad ( 1 )$$

In above equation, i,j=a, b, c, d, e, f, or g, refer to methanol, ethanol, 1-propanol, 2-pentanol, acetone, pentane, and hexane, respectively. The distances resulted from the interaction of each pair of analytes (from seven examined analytes) are listed in TABLES 5 and 6 for Detectors O and X, respectively. As it is can be seen in FIG. 22B, ethanol cluster shows some overlaps with acetone cluster in the case of Detector O. This is also confirmed from the related number to ethanol-acetone pair in TABLE 5, where the mean distance is small (2.91) which shows less selectivity compared to the same element for ethanol-acetone pair in TABLE 6 (for Detector X) which is 4.23. This is ˜45% more than that obtained for Detector O. The largest mean distance in both tables is for methanol-hexane pairs which is attributed to difference in their relative polarity numbers (listed in TABLE 3). In essence, methanol is the most polar and hexane is the most non-polar analyte tested among all the tested analytes. Moreover, the average of numbers listed in TABLE 6 for Detector X is 12.45 which is ˜43% more than the average of mean distance listed in TABLE 5 for Detector O (8.70).

TABLE 5
The Euclidean distances between the average feature vectors in the
feature space for Detector O (coated with Cr, Au, and Parylene C).
The average of all Euclidean distances in this table is 8.70.

| | a: [symbol] | b: [symbol] | c: [symbol] | d: [symbol] | e: [symbol] | f: [symbol] | g: [symbol] |
|---|---|---|---|---|---|---|---|
| a: [symbol] | 0.00 | 4.65 | 9.12 | 15.94 | 7.81 | 14.36 | 24.91 |
| b: [symbol] | 4.65 | 0.00 | 4.45 | 11.29 | 2.91 | 10.00 | 20.62 |
| c: [symbol] | 9.12 | 4.45 | 0.00 | 6.83 | 1.34 | 5.89 | 16.42 |
| d: [symbol] | 15.94 | 11.29 | 6.83 | 0.00 | 8.13 | 3.34 | 10.37 |
| e: [symbol] | 7.81 | 2.91 | 1.34 | 8.13 | 0.00 | 6.81 | 17.42 |
| f: [symbol] | 14.36 | 10.00 | 5.89 | 3.34 | 6.81 | 0.00 | 10.62 |
| g: [symbol] | 24.91 | 20.62 | 16.42 | 10.37 | 17.42 | 10.62 | 0.00 |

TABLE 6
The Euclidean distances between the average feature
vectors in the feature space for Detector X (coated
with Cr, Au, Parylene C, and Cytonix). The average
of all Euclidean distances in this table is 12.45.

| | a: [symbol] | b: [symbol] | c: [symbol] | d: [symbol] | e: [symbol] | f: [symbol] | g: [symbol] |
|---|---|---|---|---|---|---|---|
| a: [symbol] | 0.00 | 8.78 | 11.00 | 25.33 | 10.12 | 22.09 | 28.92 |
| b: [symbol] | 8.78 | 0.00 | 10.82 | 23.25 | 4.23 | 21.42 | 28.64 |
| c: [symbol] | 11.00 | 10.82 | 0.00 | 14.33 | 1.38 | 11.35 | 18.45 |
| d: [symbol] | 25.33 | 23.25 | 14.33 | 0.00 | 15.26 | 4.53 | 7.12 |
| e: [symbol] | 10.12 | 4.23 | 1.38 | 15.26 | 0.00 | 12.00 | 18.96 |
| f: [symbol] | 22.09 | 21.42 | 11.35 | 4.53 | 12.00 | 0.00 | 7.22 |
| g: [symbol] | 28.92 | 28.64 | 18.45 | 7.12 | 18.96 | 7.22 | 0.00 |

Effects of Channel Coating and Analyte Polarity.

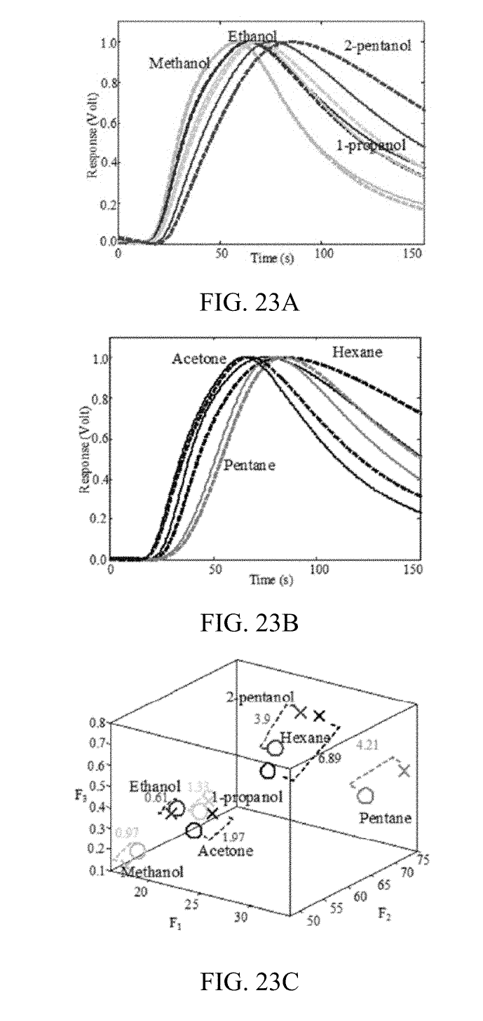

After comparing Detectors O and X in terms of their selectivity between different analytes, an evaluation of how changes in the polarity of the coating layer influences the temporal responses of the sensor to polar and non-polar analytes. In other words, the normalized temporal responses of two sensors to the same target gas, were compared, to see the effect of the channel and analyte polarities and their interaction (dipole-dipole interaction between the analyte and channel surface). The normalized transient responses of the two detectors to polar and non-polar analytes, are shown in FIGS. 23A and 23B, respectively. The extracted features from each normalized response for Detectors O and X, are represented in FIG. 23C. As shown in FIG. 23C, the order of feature vectors in the feature space changes by moving from polar analytes to non-polar ones for the two detectors. This can also be seen in the temporal responses (FIG. 23C). Detector O (with higher polarity) shows less resistance to non-polar analytes compared to Detector X (see FIG. 23A). For instance, it can be seen in FIG. 23A that diffuse-in and -out processes for methanol happen slightly faster in Detector X with less polarity compared to the slopes of diffuse-in and -out process for Detector O with higher polarity, On the other hand, for non-polar analytes such as hexane, this order changes in the temporal responses of the two detectors, where Detector O with higher polarity (e.g. the black solid line for hexane) shows faster diffusion-in and -out and eventually faster retention time compared to Detector X with less polarity of the channel surface material (e.g. the black dash line for hexane). This is due to “like dissolves like” principle: the channel surface with higher polarity (Detector O) shows a higher adsorption rate from the polar gases; whereas the channel with lower polarity (Detector X) shows a higher adsorption rate from the non-polar analytes. As a result, if the polarity of the channel coating material and compound are similar, the retention time increases (physisorption increases), as the compound interacts stronger with the channel surface. Therefore, polar compounds have long retention times on polar channels and shorter retention times on non-polar channels.

Changing the channel coating from Detector O to X (more polar to less polar) has insignificant effects on polar analytes, especially on the ones with a smaller hydro-carbon chain and higher polarity. Among the four tested alcohols, 2-pentanol (least polar alcohol) shows the largest difference in the temporal responses of the two sensors (see FIG. 23A), which means changing the channel polarity affects the polar analytes less. On the other hand, each of the two Detectors responded differently to less non polar gases such as acetone and alkanes (e.g. pentane and hexane), respectively (see FIG. 23B). This has also been projected in the feature space, where the feature vectors of Detector O (presented with O markers) and the feature vectors of Detector X (presented with X markers) and their 3D Euclidean distances are shown. As it can be seen, the distances between the feature vectors of the two Detectors in response to the non-polar gases are larger (e.g. 6.89 for hexane) as compared to the polar ones (e.g. 0.61 for ethanol). Therefore, the results shown in FIGS. 23A-23C show larger differences between the two fabricated detectors in response to the non-polar gases as compared to the polar ones. This is attributed to the higher diffusion coefficient of polar gases (TABLE 4) which makes the diffusion part of diffusion-physisorption to be more effective. In other words, for the polar gases, diffusion is the dominant term in the diffusion-physisorption equation, which makes the effect of channel coating (which has more influence on adsorption) less significant. On the other hand, the non-polar gases with lower diffusion coefficients have more time to interact with the channel surfaces, and hence, are more influenced by the channel surface material. Although diffusion rates of different gases are a significant parameter in device discrimination ability to distinguish different analytes, it is not the only parameter involved. For example, ethanol and acetone have similar diffusion coefficients (˜0.11 cm²/s), Therefore, if the diffusion rate was the only parameter for discriminating these two gases, the two detectors should have shown the same responses against these two gases and fail to distinguish between them. However, as it can be seen from FIGS. 22A-22D, Detectors O and X can distinguish between these two gases. Moreover, as it can be seen from FIGS. 23A-23C, the two detectors show a more significant difference against acetone (1.97) rather than ethanol (0.61). This is also related to their polarity and the fact that changing the channel coating has more influence on less polar gases (such as acetone) rather than polar ones (such as ethanol). This is an obvious indication of the fact that the analyte discrimination in microfluidic gas detectors is not a purely diffusion-based process, and there are analyte/channel surface-related parameters involved in enhancing/impeding sensor selectivity. As indicated in FIG. 23C, the difference between the feature vectors of 2-pentanol is 3.9, which is the largest among all the other alcohols and it is even higher than some of the less polar gases (such as acetone for which the difference between the feature vectors is 1.97). Comparing the diffusion coefficient of these two gases also justifies these numbers: acetone has a higher diffusion rate than 2-pentanol. In the next section, the surface free energy of the two fabricated channels (O and X) are estimated to quantify the interaction between the analyte and channel coating and its relation to the sensor discrimination power.

Channel Surface Free Energy.

To determine the channel surface free energy of the two fabricated detectors, Owens, Wendt, Rabel and Kaelble (OWRK) method [34] is used. The contact angle values of five of the tested analytes (methanol, ethanol, acetone, pentane, and hexane (as the representatives of the three families of alcohol, ketone and alkane)) on the channel surface of the two fabricated detectors were measured and listed in TABLE 7. The values represent the average of five measurements and the error presents the standard deviation.

TABLE 7
The contact angle measurement of five analytes on the
surfaces of both detectors. The angles listed here are the averages of five measurements and
the error represents the standard deviation. The liquid-vapor (γ_{LV}) and solid-vapor
(γ_{SV}) measured for both detectors are also listed here (the method of calculation
of these values are explained at the end of this section).

| | Contact angle on | Contact angle on | | |
|---|---|---|---|---|
| | Detector O channel | Detector X channel | | |
| Analyte | surface | surface | γ_{SL} for Detector O | γ_{SL} for Detector X |
| Methanol | 13° ± 2 | 46° ± 3 | 0.28 ± 0.07 | 0.85 ± 0.1 |
| Ethanol | 16° ± 2 | 48° ± 2 | 2.07 ± 0.1 | 2.67 ± 0.09 |
| Acetone | 5° ± 1 | 46° ± 3 | 1.37 ± 0.06 | 0.09 ± 0.1 |
| Pentane | 10° ± 2 | 17° ± 1 | 5.11 ± 0.09 | 2.07 ± 0.06 |
| Hexane | 8° ± 1 | 16° ± 1 | 7.45 ± 0.06 | 0.08 ± 0.05 |

Based on the OWRK method, each of the interfacial tensions (liquid-vapor (γ_{LV}) and solid-vapor (γ_{SV})) are broken down into two terms: polar surface tension (γ^{p}) and dispersive surface tension (γ^{d}) parts [35] (see Eq. (2) and (3)).

γ_{LV}=γ_{LV}^{d}+γ_{LV}^{p} (2)

γ_{SV}=γ_{SV}^{d}+γ_{SV}^{p} (3)

The values for polar and dispersive liquid-vapor (γ_{LV}) for the tested analytes are listed in TABLE 3. Combining Good's and Young's equations (Eqs. (4)) and substituting Eq. (2) into it will result in Eq. (5):

$$\begin{matrix} \begin{matrix}{γ}_{SL} = {γ}_{SV} + {γ}_{LV} - 2 \sqrt{{γ}_{SV}^{d} {γ}_{LV}^{d}} - 2 \sqrt{{γ}_{SV}^{p} {γ}_{LV}^{p}} \\ {γ}_{SL} = {γ}_{SV} - {γ}_{LV} cos θ\end{matrix} \} & ( 4 ) \\ ( 1 + cos θ ) ( ( {γ}_{LV}^{p} + {γ}_{LV}^{d} ) / 2 \sqrt{{γ}_{LV}^{d}} ) = \sqrt{{γ}_{SV}^{d}} + \sqrt{{γ}_{SV}^{p}} \cdot \sqrt{{γ}_{LV}^{p} / {γ}_{LV}^{d}} & ( 5 )\end{matrix}$$

This equation can be simplified to a linear equation in the form of y=A+Bx, where

$$\begin{matrix}y = ( 1 + cos θ ) ( ( {γ}_{LV}^{p} + {γ}_{LV}^{d} ) / 2 \sqrt{{γ}_{LV}^{d}} ) \\ x = \sqrt{{γ}_{LV}^{p} / {γ}_{LV}^{d}} \\ A = \sqrt{{γ}_{SV}^{d}} \\ B = \sqrt{{γ}_{SV}^{p}}\end{matrix} \} \qquad ( 6 )$$

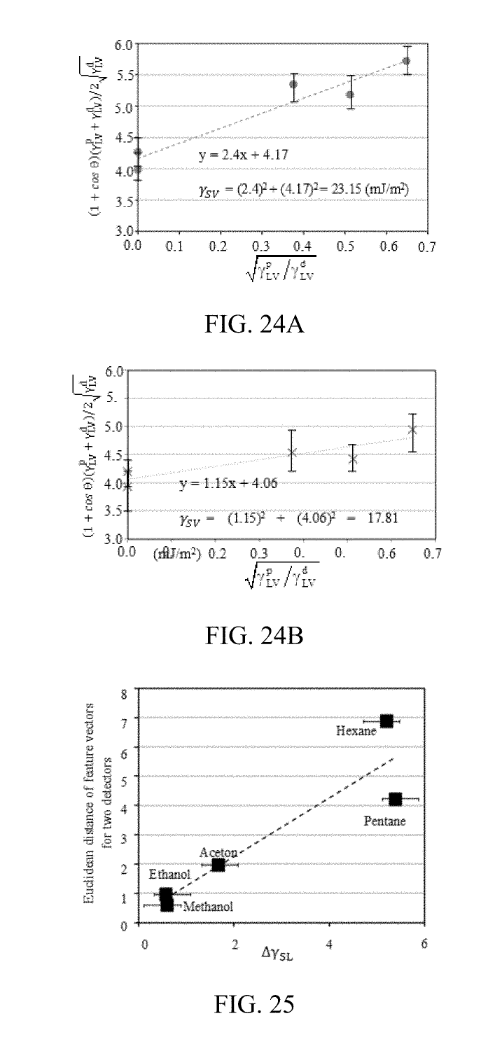

After measuring the contact angles of different analytes on the both channel surfaces of Detectors O and X, the linear Eq. (5) is used to determine the solid surface tension of each of the fabricated channels. The results are shown in FIGS. 24A and 24B for Detectors O and X, respectively. Each O or X marker in FIGS. 24A and 24B presents the average value obtained from the five runs of contact angle measurements for each analyte. The error bars present the standard deviation from the average. The solid-vapor surface tension (γ_{SV}) can then be measured from FIGS. 24A and 24B for each particular surface. In essence, the line intercept (A) and slope (B) are the square roots of the dispersive and polar parts of the solid-vapor surface tensions, respectively. The results show that the solid-vapor surface tension (γ_{SV}) for the channel surface of Detector O (coated with Parylene C as the top layer) is 23.15 mJ/m², and for the channel surface of Detector X (coated with Cytonix as top layer) is 17.81 mJ/m².

Using the Young's equation (Eq. (4)), the solid-liquid surface tensions (γ_{SL}) can then be estimated for each of the channel surfaces for different analytes. These results are listed in TABLE 7. Interestingly, the differences between the values of γ_{SL} for the two surfaces (Detectors O and X) are smaller for polar analytes (e.g. for methanol it is 0.56) and higher for non-polar analytes (e.g. for hexane it is 5.2). This was also observed in FIG. 23C, where the feature vectors of non-polar gases showed greater Euclidean distances for the two detectors, whereas the feature vectors for polar gases for the two detectors showed smaller Euclidean distances in the feature space. FIG. 25 shows the linear relation between the distances of the feature vectors of the two detectors (shown in FIG. 23C) vs. the differences between γ_{SL} for the two channel surfaces of the two detectors (Δγ_{SL}) for each of five tested analytes. This also shows as the surface of the channel changes the non-polar gases behave more differently than the polar ones. This may be attributed to the fact that for the non-polar gases (with smaller diffusion rates) physisorption of the gas molecules to the channel walls is more dominant. As a result, the response of the Detector X against the non-polar gases is more than that of Detector O.

Two microfluidic-based gas detectors were fabricated with two different channel coating combinations (of layers) with different hydrophobicity. The selectivity of the two fabricated detectors among different analytes including: alcohols, ketones, and alkanes, were compared (both qualitatively and quantitatively) using a feature extraction method. The feature space presents that Detector O (coated with Cytonix) has a better segregation power among the tested analytes compared to Detector X. It has been shown that changing the polarity of the channel coating creates a more significant effect on the position of feature vectors of non-polar gases compared to polar ones. This is attributed to the higher diffusion rates of polar gases as compared to non-polar ones. This means that for the polar gases diffusion is the dominant term in the diffusion-physisorption equation, which makes the effect of channel coating (which has more influence on adsorption) less significant. On the other hand, for the non-polar gases, lower diffusion coefficients result in having more time to interact with the channel surfaces, and hence, those are more influenced with the channel surface material, The comparison between the surface tensions of both channels showed that the difference in the solid-liquid surface for non-polar analytes is greater compared to polar ones. This supports the fact that changing the polarity of the channel coating alters more significantly the position of the feature vectors for non-polar analytes. These results show that when it comes to selecting the best channel surface coating material, the choice of non-polar coating surfaces offer more selectivity against non-polar gases, and in the case of polar gases this coating has less effects. This can be used to design an array of micro-channels with different polarities to increase the segregation power of the device.

Although embodiments described herein have been described in some detail by way of illustration and example for the purposes of clarity of understanding, it will be readily apparent to those of skill in the art in light of the teachings described herein that changes and modifications may be made thereto without departing from the spirit or scope of the appended claims. Such modifications include the substitution of known equivalents for any aspect of the invention in order to achieve the same result in substantially the same way. Numeric ranges are inclusive of the numbers defining the range. The word “comprising” is used herein as an open ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as herein described and with reference to the figures.

### REFERENCES

[1] M. Bunge, et al. “On-line monitoring of microbial volatile metabolites by proton transfer reaction-mass spectrometry.” Applied and environmental microbiology, vol. 74.7, pp. 2179-2186, 2008.
[2] F. Hossein-Babaei, and V. Ghafarinia. “Gas analysis by monitoring molecular diffusion in a microfluidic channel.” Analytical chemistry, vol. 82.19, pp. 8349-8355, 2010.
[3] Amorim, and Z. L. Cardeal, “Breath air analysis and its use as a biomarker in biological monitoring of occupational and environmental exposure to chemical agents,” Journal of Chromatography B, vol. 853, pp. 1-8, 2008.
[4] Xie, Yi, et al. “Three-dimensional ordered ZnO—CuO inverse opals toward low concentration acetone detection for exhaled breath sensing,” Sensors and Actuators B: Chemical, vol. 21, pp. 255-262, 2015.
[5] M. Philips, N. Altorki, J. Austin, R. Cameron, J. Greenberg, R. Kloss, R. Maxfield, M. Munawar, and H. Pass, “Prediction of lung cancer using volatile biomarkers in breath,” Cancer Biomarkers, vol. 3, no. 2, pp. 95-109, 2007.
[6] L. Zhu, et al. “Integrated microfluidic gas sensor for detection of volatile organic compounds in water” Sensors and Actuators B: Chemical, vol. 121.2 pp. 679-688, 2007.
[7] S. Zampolli, et al. “Real-time monitoring of sub-ppb concentrations of aromatic volatiles with a MEMS-enabled miniaturized gas-chromatograph.” Sensors and Actuators B: Chemical, vol. 141.1, pp. 322-328. 2009.
[8] A. W. Boots, et al. “Identification of microorganisms based on headspace analysis of volatile organic compounds by gas chromatography-mass spectrometry.” Journal of breath research, vol. 8.2, pp. 027106, 2014.
[9] A. Garg, et al. “Zebra GC: A mini gas chromatography system for trace-level determination of hazardous air pollutants.” Sensors and Actuators B: Chemical, vol. 212, pp. 145-154, 2015.
[10] L. Li, et al. “Mini 12, Miniature Mass Spectrometer for Clinical and Other Applications, Introduction and Characterization.” Analytical chemistry, vol. 86.6 pp. 2909-2916, 2014.
[11] W. F. Karasek, and R. E. Clement “Basic gas chromatography-mass spectrometry: principles and techniques” Elsevier, 20120
[12] J. W. Gardner, and P. N. Bartlett. “A brief history of electronic noses.” Sensors and Actuators B: Chemical, vol. 18.1, pp. 210-211, 1994.
[13] K. Arshak, et al. “A review of gas sensors employed in electronic nose applications.” Sensor review, vol. 24.2, pp. 181-198, 2004.
[14] M. Holmberg, et al. “Drift counteraction for an electronic nose.” Sensors and Actuators B: Chemical 36.1, pp. 528-535, 1996.
[15] W. J. Harper, “The strengths and weaknesses of the electronic nose.” Headspace analysis of foods and flavors. Springer US, pp. 59-71, 2001.
[16] F. Hossein-Babaei and V. Ghafarinia, “Compensation for the drift-like terms caused by environmental fluctuations in the responses of chemoresistive gas sensors,” Sensors and Actuators B, vol. 143, pp. 641-648, 2010.
[17] F. Hossein-Babaei, and A. Amini. “Recognition of complex odors with a single generic tin oxide gas sensor.” Sensors and Actuators B: Chemical, vol. 194, pp. 156-163, 2014.
[18] F. Hossein-Babaei, M. Hemmati, and M. Dehmobed. “Gas diagnosis by a quantitative assessment of the transient response of a capillary-attached gas sensor.” Sensors and Actuators B: Chemical, vol. 107.1, pp. 461-467, 2005.
[19] M. Paknahad, V. Ghafarinia, and F. Hossein-Babaei. “A microfluidic gas analyzer for selective detection of biomarker gases” Sensors Applications Symposium (SAS), 2012 IEEE, pp. 1-5. IEEE, 2012.
[20] F. Hossein-Babaei, M. Paknahad, and V. Ghafarinia, “A miniature gas analyzer made by integrating a microchannel with a chemoresistor,” Lab-on-a-Chip, vol. 12, pp. 1874-1880, 2012.
[21] V. Ghafarinia, A. Amini, and M. Paknahad. “Gas identification by a single gas sensor equipped with microfluidic channels.” Sensor Letters, vol. 10.3-4, pp. 845-849, 2012.
[22] M. Paknahad, Mohammad, et al. “Highly selective multi-target 3D-printed microfluidic-based breath analyzer.” 2016 IEEE 29th International Conference on Micro Electro Mechanical Systems (MEMS). IEEE, pp. 905-908, 2016.
[23] N. Yamazoe, G. Sakai, and K. Shimanoe, “Oxide semiconductor gas sensors.” Catalysis Surveys from Asia, vol. 7.1, pp. 63-75, 2003.
[24] C. L. Yaws, “Chemical properties handbook.”, McGraw Hill Professional, 1998.
[25] Binh-Khiem, Nguyen, Kiyoshi Matsumoto, and Isao Shimoyama. “Porous Parylene and effects of liquid on Parylene films deposited on liquid.” Micro Electro Mechanical Systems (MEMS), 2011 IEEE 24th International Conference on. IEEE, 2011.
[26] H. Noh, P. J. Hesketh, and G. C. Frye-Mason. “Parylene gas chromatographic column for rapid thermal cycling.” Journal of Microelectromechanical Systems, vol. 11.6, pp. 718-725, 2002.
O. Grinberg, et al. “Antibiotic nanoparticles embedded into the Parylene C layer as a new method to prevent medical device-associated infections.” Journal of Materials Chemistry B, vol. 3.1, pp. 59-64, 2015.
F. Hossein-Babaei, and S. Rahbarpour. “Alteration of pore size distribution by sol-gel impregnation for dynamic range and sensitivity adjustment in Kelvin condensation based humidity sensors.” Sensors and Actuators B: Chemical, vol. 191, pp. 572-578, 2014.
[29] F. Hossein-Babaei “Novel Device and Method for Gas Analysis” Canadian Patent 2,395,563.
[30] F. Hossein-Babaei, M. Paknahad, and V. Ghafarinia, Lab on a Chip 12, 1874-1880 (2012).
[31] M. Paknahad, J. S. Bachhal, A. Ahmadi & M. Hoorfar, IEEE MEMS, pp. 905-908, (2016).
[32] W. Cao, and Y. Duan, Clinical chemistry 52, 800, (2006). 4.
[33] J. W. Gardner, H. Woo Shin, and E. L. Hines., Sensors and Actuators B: Chemical, 70, 19, (2000).
[34] Żenkiewicz, M. Methods for the calculation of surface free energy of solids. Journal of Achievements in Materials and Manufacturing Engineering 2007, 24.1, 137-145.
[35] Kaelble, D. H. Dispersion-polar surface tension properties of organic solids. The Journal of Adhesion 1970, 2.2, 66-81.
[36] Syed, J. A.; Tang, S.; Meng, X. Super-hydrophobic multilayer coatings with layer number tuned swapping in surface wettability and redox catalytic anti-corrosion application. Scientific Reports, 2017.
[37] Haynes, W. M. CRC handbook of chemistry and physics. CRC press, 2014.
[38] Paknahad, M.; Bachhal, J. S.; Ahmadi, A.; Hoorfar, M. Characterization of channel coating and dimensions of microfluidic-based gas detectors. Sensors and Actuators B: Chemical 2016, 241, 55-64.

What is claimed is:

1. A gas detection apparatus, the apparatus comprising:
(a) a channel having an inner surface and having at least one opening, such that the channel is optionally in fluid communication with a sample gas when the opening is in an open position and optionally having a closed position, the inner surface having a coating comprising:
(i) a first layer comprising a non-reactive metal or non-reactive metalloid compound; and
(ii) a second layer comprising a moisture barrier; and
(b) a gas sensor disposed within the channel.

2. The apparatus of claim 1, wherein the second layer comprising a moisture barrier has a gas permeability sufficient to absorb the gas particles being sampled.

3. The apparatus of claim 1, wherein:
(i) the non-reactive metal is selected from one or more of the following: copper; chromium; ruthenium; rhodium; palladium; gold; silver; osmium; iridium; platinum; titanium; niobium; tantalum; bismuth; tungsten; tin; nickel; cobalt; manganese; and zinc; or
(ii) is metalloid compound is SiO₂.

4. The apparatus of claim 1, wherein the moisture barrier with high porosity is Parylene or Polydimethylsiloxane (PDMS).

5. The apparatus of claim 4, wherein the Parylene is selected from Parylene C, Parylene N or Parylene D.

6. The apparatus of claim 5, wherein the Parylene is Parylene C.

7. The apparatus of claim 1, wherein the non-reactive metal is selected from one or more of the following: copper; chromium; ruthenium; rhodium; palladium; gold; silver; iridium; platinum; titanium; niobium; and tantalum.

8. The apparatus of claim 1, wherein the coating is chromium, gold and Parylene C.

9. The apparatus of claim 1, wherein the channel further comprises a heater.

10. The apparatus of claim 9, wherein the heater is operable to increase the channel temperature to at least 80° C.

11. The apparatus of claim 1, wherein the gas sensor is selected from one or more of the following: an infra-red (IR) sensor; a chemoresistive sensor; an electrochemical sensor; an optical sensor; a capacitive sensor; a semiconductor sensor; an acoustical sensor; a thermoelectric sensor; and a combination thereof.

12. The apparatus of claim 1, wherein the gas sensor is a semiconductor sensor.

13. The apparatus of claim 1, wherein the gas sensor is a Metal Oxide Semiconductor (MOS).

14. The apparatus of claim 1, wherein the gas sensor is a tin oxide-based chemoresistive gas sensor.

15. The apparatus of claim 1, wherein there is more than one gas sensor in the channel.

16. The apparatus of claim 1, wherein the channel length to channel depth ration is 150:1.

17. The apparatus of claim 1, wherein the channel width to channel depth ration is 3:1.

18. The apparatus of claim 1, wherein the channel length is 3 mm wide, 30 mm long and 200 μm deep.

19. The apparatus of claim 1, wherein the first layer comprises chromium and gold.

20. The apparatus of claim 19, wherein the chromium was applied to the channel prior to the gold.

21. The apparatus of claim 19, wherein the second layer comprises Parylene C.

22. The apparatus of claim 1, wherein the first layer comprises SiO₂.

23. The apparatus of claim 22, wherein the second layer comprises Parylene C.

24. The apparatus of claim 1, wherein the opening further comprises a closed position.

25. The apparatus of claim 1, wherein the apparatus further comprises a second opening.

26. The apparatus of claim 25, wherein the second opening has both an open and closed position.

27. The apparatus of claim 1, wherein the apparatus further comprises a liquid trap positioned in fluid communication with the at least one opening.

28. The apparatus of claim 1, wherein the apparatus further comprises a humidity filter positioned in fluid communication with the at least one opening.

29. The apparatus of claim 1, wherein the apparatus further comprises a pump, which is optionally in fluid communication with the at least one opening.

30. The apparatus of claim 25, wherein the apparatus further comprises a pump, which is optionally in fluid communication with the second opening.

31. The apparatus of claim, wherein the apparatus further comprises a compressed air source, which is optionally in fluid communication with the channel.

32. The apparatus of claim 1, wherein the apparatus further comprises a compressed gas source, which is optionally in fluid communication with the channel.

33. The apparatus of claim 1, wherein the apparatus further comprises a pentane plume, which is optionally in fluid communication with the channel.

34. The apparatus of claim 1, wherein the apparatus further comprises a compressed O₂ source or N₂ source or separate O₂ and N₂ sources, which are optionally in fluid communication with the channel.

35. The apparatus of claim 1, wherein the apparatus further comprises a cleaning solution, which is optionally in fluid communication with the channel.

36. The apparatus of claim 32, wherein the compressed gas source is selected from one or more of the following: air; CO₂; O₂; or N₂.

37. The apparatus of claim 32, wherein there is more than one compressed gas source, selected from the following: air; CO₂; O₂; or N₂.

38. The apparatus of claim 1, wherein the apparatus further comprises a heater for heating the channel.

39. The apparatus of claim 38, wherein the heater is selected from the following: a wire; a sputtered electrodes; a heating pad; an optical heater; a microwave heater; an electromagnetic heater; and combinations thereof.

40. The apparatus of claim 1, wherein the second layer comprises Parylene C and Cytonix.

41. The apparatus of claim 40, wherein the Parylene C was applied to the channel prior to the Cytonix.

42. The apparatus of claim 1, wherein the coating is:
(a) chromium; (b) gold; (c) Parylene C; and Cytonix.

43. The apparatus of claim 1, wherein the channel has non-polar coating when used for non-polar analytes.
